(19) United States
(12) Patent Application Publication
(10) Pub. No.: US 2022/0259637 A1
(43) Pub. Date: Aug. 18, 2022

(54) METHOD FOR ENRICHING NUCLEIC ACIDS BY SIZE

(71) Applicant: QIAGEN GmbH, Hilden (DE)

(72) Inventors: Lothar BREITKOPF, Hilden (DE); Jasper STROEDER, Hilden (DE); Markus SPRENGER-HAUSSELS, Hilden (DE)

(21) Appl. No.: 17/620,612

(22) PCT Filed: Jun. 26, 2020
(86) PCT No.: PCT/EP2020/068080

§ 371 Date: Dec. 17, 2021

(30) Foreign Application Priority Data
Jun. 28, 2019 (EP) 19183297.1

(51) Int. Cl. C12Q 1/6806; C12Q 1/686; C12N 15/10; C12N 15/82

(52) U.S. Cl. CPC C12Q 1/6806; C12N 15/8206; C12N 15/1013; C12Q 1/686

(57) ABSTRACT

The present invention provides a poly(alkylene oxide) polymer based size selective method for enriching nucleic acid molecules having a length below a cut-off value from a nucleic acid containing sample, the method comprising: (a) preparing a binding mixture comprising —the nucleic acid containing sample, —a poly(alkylene oxide) polymer and —a salt and binding nucleic acid molecules of different sizes to a solid phase which comprises a functional group, preferably carboxylated magnetic particles; (b) separating the solid phase with the bound nucleic acid molecules from the remaining sample; and (c) contacting the solid phase with the bound nucleic acid molecules at least once with an elution composition comprising a poly(alkylene oxide) polymer and a salt to selectively elute nucleic acid molecules having a length below the cut-off value from the solid phase while larger nucleic acid molecules having a length above the cut-off value remain bound to the solid phase, wherein the concentration (w/v) of the poly(alkylene oxide) polymer in the elution composition is lower than the concentration (w/v) of the poly(alkylene oxide) polymer in the binding mixture of (a); (d) separating the solid phase with the bound larger nucleic acid molecules from the eluted nucleic acid molecules; and (e) optionally further purifying the eluted nucleic acid molecules. The method is particularly useful for separating extracellular target nucleic acids by size.

### FIELD OF INVENTION

The present invention provides a method for enriching nucleic acid molecules having a length below a cut-off value from a nucleic acid containing sample. The method is particularly useful for enriching extracellular nucleic acid molecules, such as circulating cell free DNA (ccfDNA), from cell-free or cell-depleted body fluid samples. Furthermore, kits suitable for performing the method are provided.

### BACKGROUND OF THE INVENTION

Different methods for isolating nucleic acids are well-known in the prior art. If it is intended to isolate a specific nucleic acid of interest from other nucleic acids, the separation process is usually based on differences in parameters of the target and the non-target nucleic acid such as for example their topology (for example super-coiled DNA from linear DNA), their length (size) or chemical differences (e.g. DNA from RNA) and the like. For certain applications, the difference in the length is an important criterion to distinguish target nucleic acids from non-target nucleic acids, e.g. in the field of extracellular nucleic acids. Most of the nucleic acids in the body are located within cells, but a small amount of nucleic acids can also be found circulating freely in human body fluids (commonly referred to as extracellular, cell-free or circulating cell-free DNA/RNA (ccfDNA/RNA)). These extracellular nucleic acids inter alia enter the body fluids by cell necrosis, apoptosis or active secretion by healthy and diseased cells. Extracellular nucleic acids have been identified in blood, plasma, serum and other body fluids. Extracellular nucleic acids that are found in respective samples are to a certain extent degradation resistant due to the fact that they are protected from nucleases (e.g. because they are secreted in form of a proteolipid complex, are associated with proteins or are contained in vesicles). The presence of elevated levels of extracellular nucleic acids such as DNA and/or RNA in many medical conditions, malignancies, and infectious processes is of interest inter alia for screening, diagnosis, prognosis, surveillance for disease progression, for identifying potential therapeutic targets, and for monitoring treatment response. Additionally, elevated fetal DNA/RNA in maternal blood is being used to determine e.g. gender identity, assess chromosomal abnormalities, and monitor pregnancy-associated complications. Thus, extracellular nucleic acids are in particular useful in non-invasive diagnosis and prognosis and can be used e.g. as diagnostic markers in many fields of application, such as non-invasive prenatal genetic testing, oncology, transplantation medicine or many other diseases and, hence, are of diagnostic relevance (e.g. fetal- or tumor-derived nucleic acids). However, extracellular nucleic acids are also found in healthy human beings. Common applications and analysis methods of extracellular nucleic acids are e.g. described in WO97/035589, WO97/34015, Swarup et al, FEBS Letters 581 (2007) 795-799, Fleischhacker Ann. N.Y. Acad. Sci. 1075: 40-49 (2006), Fleischhacker and Schmidt, Biochmica et Biophysica Acta 1775 (2007) 191-232, Hromadnikova et al (2006) DNA and Cell biology, Volume 25, Number 11 pp 635-640; Fan et al (2010) Clinical Chemistry 56:8.

Extracellular nucleic acids are usually only comprised in a low concentration in the samples. E.g. free circulating nucleic acids are present in plasma in a concentration of 1-100 ng/ml plasma. Furthermore, extracellular nucleic acids often circulate as fragments of a length of 600 nt, such as 500 nt (circulating nucleosomes). For extracellular DNA in plasma, the average length is often only approximately 130-170 bp. Frequently, also multiples of this length are observed as the mentioned fragments, wherein the multiples can have approximate lengths of 320-360 bp or 490-530 bp. As these repeated lengths correspond approximately to a single nucleosome size and multiplies thereof, one may expect that the patterns of DNA cleavage are guided by nucleosome positioning. The size pattern of the small extracellular DNA fractions has been correlated to mono-, di- and tri-nucleosomal structures that stem from the cleavage of chromatin DNA via endogenous endonucleases in apoptotic cells.

In addition to small extracellular DNA species 600 nt extracellular DNA also comprises high molecular weight (HMW) species including fragments of 5,000 nt or 10,000 nt. The presence of HMW species in body fluids has been inter alia attributed to necrotic processes. The amount of such high molecular weight species in the extracellular fraction can significantly increase upon storage of cell-containing body fluids (such as blood or urine), because dying cells release genomic DNA. For isolating extracellular nucleic acids from a body fluid, cells are usually removed to prepare a cell-free or cell-depleted body fluid sample (e.g. blood plasma, cell-free urine) which comprises the extracellular nucleic acids. High molecular weight nucleic acids which may e.g. be released during storage remain in the extracellular fraction, whereby the smaller extracellular nucleic acids become contaminated and diluted with such high molecular nucleic acids. For many applications, it is desirous to remove the high molecular weight nucleic acids from the smaller extracellular nucleic acids, so as to obtain the smaller extracellular nucleic acids as separate, enriched fraction from which larger nucleic acids (e.g. of 500 nt or 600 nt) have been depleted. Such approach is e.g. described in EP 1 524 321.

Several approaches exist in order to isolate DNA of a specific target size, respectively of a specific target size range. A classic method for isolating DNA of a target size involves the separation of the DNA in a gel, cutting out the desired gel band(s) and then isolating the DNA of the target size from the gel fragment(s). However, respective methods are time consuming, as the portion of the gel containing the nucleic acids of interest must be manually cut out and then treated to degrade the gel or otherwise extract the DNA of the target size from the gel slice. Another technology is the size selective precipitation with a poly(alkylene oxide) polymer containing buffer, in particular polyethylene glycol based buffers (Lis and Schleif Nucleic Acids Res. 1975 March; 2(3):383-9) or the binding/precipitation on carboxyl-functionalized beads (DeAngelis et al, Nuc. Acid. Res. 1995, Vol 23(22), 4742-3; U.S. Pat. Nos. 5,898,071 and 5,705,628 and 6,534,262). There is a need for further efficient and advantageous methods for enriching small target nucleic acid molecules, such as target extracellular DNA, based on their size.

It is an object of the present invention to provide a method for enriching nucleic acid molecules of a target size from a nucleic acid containing sample which comprises nucleic acid molecules of different sizes. It is furthermore an object of the present invention to provide a method that allows to enrich extracellular nucleic acids (such as extracellular DNA molecules) having a length below a cut-off value from a cell-free or cell-depleted body fluid sample, while efficiently depleting high molecular weight species. It is also an object to provide a method that allows to enrich nucleic acid molecules of different sizes as separate fractions.

### SUMMARY OF THE INVENTION

The present invention provides an advantageous size selective nucleic acid enrichment method that is particularly suitable for size-selectively separating extracellular nucleic acids molecules, such as extracellular DNA of a certain size/size range, as target nucleic acid molecules from a cell-free or cell-depleted body fluid sample.

According to a first aspect, a poly(alkylene oxide) polymer based size selective method is provided for enriching nucleic acid molecules having a length below a cut-off value from a nucleic acid containing sample, the method comprising:
(a) preparing a binding mixture comprising
   the nucleic acid containing sample,
   a poly(alkylene oxide) polymer and
   a salt
   and binding nucleic acid molecules of different sizes to a solid phase which comprises a functional group, preferably carboxyl groups;
(b) separating the solid phase with the bound nucleic acid molecules from the remaining sample; and
(c) contacting the solid phase with the bound nucleic acid molecules at least once with an elution composition comprising a poly(alkylene oxide) polymer and a salt to selectively elute smaller nucleic acid molecules having a length below the cut-off value from the solid phase while larger nucleic acid molecules having a length above the cut-off value remain bound to the solid phase, wherein the concentration (w/v) of the poly(alkylene oxide) polymer in the elution composition is lower than the concentration (w/v) of the poly(alkylene oxide) polymer in the binding mixture of (a);
(d) separating the solid phase with the bound larger nucleic acid molecules from the eluted nucleic acid molecules; and
(e) optionally further purifying the eluted nucleic acid molecules.

According to a second aspect, a method is provided for enriching target extracellular DNA molecules having a length below a cut-off value from a cell-depleted or cell-free body fluid sample, which comprises enriching target extracellular DNA molecules from the sample using the method according to the first aspect.

According to a third aspect, a kit is provided for the size selective enrichment of nucleic acid molecules, preferably extracellular DNA molecules, having a length below a cut-off value from a nucleic acid containing sample, said kit comprising
(a) a binding reagent comprising at least one poly(alkylene oxide) polymer and at least one salt;
(b) magnetic particles for binding target nucleic acid molecules in the presence of the binding reagent (a); and
(c) an elution reagent comprising at least one poly(alkylene oxide) polymer and at least one salt and/or a dilution reagent for preparing the reagent (c) by combining the dilution reagent with the binding reagent (a);
(d) optionally at least one washing solution; and
(e) optionally an elution solution,
wherein the concentration of the poly(alkylene oxide) polymer in the binding reagent (a) is higher than the concentration of the poly(alkylene oxide) polymer in the reagent (c).

A respective kit can be advantageously used in conjunction with and for performing the method according to the first and second aspect of the present disclosure.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE FIGURES

FIG. 1: The figure shows an electropherogram of cell-free DNA obtained from a blood plasma sample. Besides the fraction of the mono-, di- and trinucleosomal ccfDNA peaks (with sizes of˜170, 340, 510 bp) a high molecular weight fraction (HMW) is found in the plasma sample. The differently sized fractions can be readily distinguished.

FIG. 2: shows the electropherogram of the starting material comprised of two libraries and a linearized plasmid that resemble the DNA size distribution of isolated ccfDNA. Please note the presence of three DNA peaks in the electropherogram. A small library around 130 bp (˜55 s) a larger library with an average of 300 bp (˜70 s) and the linearized plasmid at 2.7 kb (˜105 s).

FIG. 3: shows the electropherogram of the spin-column purified supernatant after the binding of nucleic acids to magnetic particles in step (a). Please note that the DNA is highly concentrated after the spin-column purification. Visible are two prominent DNA peaks at 52 s and 105 s besides the markers at 42 s and 120 s.

FIG. 4: shows the overlay of electropherograms following the size-selective elution step (c) according to the present disclosure for mimicked ccfDNA with alternating dilution factors of the PEG buffer. Lower PEG concentrations lead to higher elution of small DNA having a length pf 130 bp and 300 bp during this step. The DNA is highly enriched after spin-column purification.

FIG. 5: Overlay of electropherograms of the final eluates comprising the longer DNA molecules that were obtained following the size selection of ccfDNA-like samples (starting material). Prior thereto, selective elution of the smaller DNA took place under varying PEG-concentrations (respectively dilution factors to generate the elution composition as indicated in the legend). The longer DNA molecules that remained bound to the solid phase were then eluted to provide the final eluates. Two visible DNA signals are present in the final eluates. A weaker DNA signal around 70 seconds and a strong peak around 105 seconds, demonstrating that the majority of the DNA molecules in the final eluate correspond to longer DNA molecules having a length above the set cut-off value.

FIG. 6: The DNA yields that were recovered by performing the method according to the present disclosure using a DNA sample that mimicked the size-distribution of ccfDNA (starting material) are shown. The colors (greyscale) represent experiments with the same starting material but varying PEG concentrations (respectively, dilution factors) during the size-selective elution steps.

FIG. 7: displays the electropherogram from the starting material used for the method according to the present disclosure with concentrated ccfDNA (starting material used in Example 3). The starting material was diluted with a factor of 1:2 prior to analysis on the bioanalyzer. Please note that the sample contains DNA of varying sizes, ranging from 52 seconds to 112 seconds.

FIG. 8: shows the spin column purified supernatants after binding with 2.0× Vol. 20% PEG 8000 (step (a) according to the method of the present disclosure). Please note that the sample is highly concentrated following the spin-column purification. Undiluted supernatant was loaded on the bioanalyzer.

FIG. 9: shows the resulting electropherogram after spin-column purification of the supernatant from the first size-selective elution step. The eluates were diluted with a factor of 1:4 prior to analysis with the bioanalyzer, indicating a high concentration of DNA. The PEG-buffer was diluted with buffer TE in a ratio of 0.6× to generate the elution composition for the size-selective elution. Please note how the smaller DNAs are predominantly eluted from the beads during this step.

FIG. 10: shows the resulting electropherogram after spin-column purifying the DNA fraction that has been eluted during the second size-selective elution step of the protocol. In this step, the PEG-buffer was diluted with buffer TE in a ratio of 0.6× to generate the elution composition. Please note how the smaller DNAs are predominantly eluted from the beads during this step, while longer nucleic acids (>500 bp) remain bound to the magnetic particles.

FIG. 11: shows the resulting electropherogram of the final eluates following the size selection workflow by selective elution according to the method of the present disclosure. Size-selective elution has been performed in advance with a PEG buffer dilution factor of 0.6× (volumes PEG-buffer to TE). Please note the enrichment of the longer nucleic acids (>500 bp) in the final eluates compared to the smaller ones in the starting material (see FIG. 7). The smaller DNA molecules were efficiently depleted in the size-selective elution steps from the beads.

FIG. 12: shows a stacked bar graph that displays the yields of each step along the size-selection method according to the present disclosure. Please note how the majority of the DNA fragments is eluted during the first size-selective elution step (corresponds to the fraction comprising small DNA molecules) or is present in the final eluate (corresponds to the fraction comprising large DNA molecules, which may be additionally recovered if desired).

FIG. 13: shows an electropherogram of the starting material used in Example 4 before nucleic acid separation by size. Peaks representing small DNA fragments (approx. 130 bp, 40 s) and large DNA fragments (approx. 300 bp-1800 bp, 55-75 s) were identified.

FIG. 14: shows an overlay of electropherograms of the spin column purified DNA from the supernatant. 1.1×sample volume of 30% (w/v) PEG-containing buffer was used to mediate the binding of predominantly all nucleic acids to the magnetic carboxylated beads.

FIG. 15: shows an overlay of electropherograms of the spin column purified DNA from the supernatant of the first size-selective elution step with different mixing ratio of 30% PEG 8000 (w/v) and buffer TE. The ratios of PEG-buffer to TE buffer that were used for the size-selective elution step are written in the legend.

FIG. 16: shows an overlay of electropherograms after final elution of DNA from carboxylated beads following size-selective elution of small DNA fragments (approx. 120 to 130 bp) with various dilutions of a PEG-buffer comprising 30% PEG before being diluted. The dilution factor of PEG-buffer to TE that was used during the size-selective elution step can be found in the legend. A higher dilution factor indicates a higher PEG concentration during this step. Notable are the differences in the small DNA fragments peak (40 s) for the various dilutions during the size-selective elution.

FIG. 17: displays the DNA concentration of the final eluates comprising the longer DNA molecules, determined by measuring with Qubit high sense dsDNA Kit. The smaller target DNA molecules were selectively eluted from the solid phase during size-selective elution step (c). The graph illustrates the working range for the size selective elution step in order to selectively elute the smaller target DNA having a length below the cut-off value while maintaining binding of the larger DNA having a length above the cut-off value. Note, that the working range depends on the size of the target DNA molecules to be enriched. The decrease in yield represents a loss of longer DNA molecules during size-selective elution of the smaller target DNA molecules.

### DETAILED DESCRIPTION OF THE INVENTION

The present invention provides a very efficient poly(alkylene oxide) polymer based size-selective method for enriching nucleic acid molecules having a length below a cut-off value from a nucleic acid containing sample by introducing a size-selective elution step. The method is particularly suitable for enriching extracellular nucleic acid molecules comprised in a cell-free or cell-depleted body fluid according to their size (length). It moreover allows a size-selective fractionation of the comprised nucleic acids, whereby different fractions comprising differently sized nucleic acids may be provided. Furthermore the present invention provides a kit for the size selective enrichment of nucleic acid molecules.

### Method

According to a first aspect of the present disclosure a poly(alkylene oxide) polymer based size selective method is provided for enriching nucleic acid molecules having a length below a cut-off value from a nucleic acid containing sample, the method comprising:
(a) preparing a binding mixture comprising
   the nucleic acid containing sample,
   a poly(alkylene oxide) polymer and
   a salt
   and binding nucleic acid molecules of different sizes to a solid phase which comprises a functional group, preferably acidic groups such as carboxyl groups;
(b) separating the solid phase with the bound nucleic acid molecules from the remaining sample; and
(c) contacting the solid phase with the bound nucleic acid molecules at least once with an elution composition comprising a poly(alkylene oxide) polymer and a salt to selectively elute smaller nucleic acid molecules having a length below the cut-off value from the solid phase while larger nucleic acid molecules having a length above the cut-off value remain bound to the solid phase, wherein the concentration (w/v) of the poly(alkylene oxide) polymer in the elution composition is lower than the concentration (w/v) of the poly(alkylene oxide) polymer in the binding mixture of (a);
(d) separating the solid phase with the bound larger nucleic acid molecules from the eluted nucleic acid molecules; and
(e) optionally further purifying the eluted nucleic acid molecules.

In said method, the nucleic acid molecules present in the binding mixture efficiently bind to the solid phase with high yield. The inventors found that nucleic acid molecules, such as extracellular DNA molecules, having a length below a cut-off value can be size-selectively eluted from the solid phase after being bound to the phase in a binding mixture. The selectively eluted nucleic acid molecules having a length below the cut-off value are thereby provided in form of an enriched fraction. The selective elution is achieved by contacting the solid phase with the bound nucleic acid molecules with an elution composition comprises a poly(alkylene oxide) polymer and a salt, wherein the concentration of the poly(alkylene oxide) polymer of the elution composition is lower than the concentration of the poly(alkylene oxide) polymer of the binding mixture. By providing such elution composition, the target nucleic acid molecules having a length below a cut-off value can advantageously be separated from the nucleic acid molecules having a size above the cut-off value in a fast and efficient manner. The method is particularly suitable for the enriching extracellular nucleic acid species such as extracellular DNA molecules by length. The cut-off value for the target nucleic acid molecules to be enriched by size can be flexibly adjusted by modifying the concentration of the poly(alkylene oxide) polymer in the elution composition. Thereby, conditions can be established that favor the binding of nucleic acid molecules having a length above the cut-off value, such as HMW species, which thus remain bound to the solid phase, while nucleic acid molecules having a length below the cut-off value such as the target extracellular DNA molecules, are efficiently eluted and thereby are enriched in the obtained eluate. The method can be easily automated and allows to separate the nucleic acid molecules in different fractions comprising nucleic acids molecules of different sizes/size ranges. The separation of different species of e.g. extracellular DNA molecules by the invented method enables analysis of different extracellular nucleic acid molecule size ranges for (diagnostic) analysis and fundamental studies (e.g. relation of extracellular nucleic acid species). Therefore, the present invention provides an important improvement compared to prior art size selective nucleic acid isolation methods.

The individual steps of the method and preferred embodiments will now be explained in detail.

### Step (a)

Step (a) comprises preparing a binding mixture comprising
the nucleic acid containing sample,
a poly(alkylene oxide) polymer and
a salt
   and binding nucleic acid molecules of different sizes to a solid phase. As is described herein, the solid phase comprises a functional group, which is preferably acidic. Particularly suitable is a carboxyl group as functional group. In an advantageous embodiment, the solid phase is provided by carboxylated magnetic particles.

The binding mixture comprises at least one poly(alkylene oxide) polymer. The contained poly(alkylene oxide) polymer, preferably a polyethylene glycol, precipitates nucleic acid (preferably DNA) molecules so that they bind to the solid phase. Under the used binding conditions, nucleic acid molecules of different sizes bind to the solid phase. Thereby, a solid phase is provided having bound thereto the nucleic acid molecules.

According to a preferred embodiment, step (a) comprises adding a binding reagent to the nucleic acid containing sample to prepare the binding mixture, wherein the binding reagent comprises the poly(alkylene oxide) polymer, preferably a polyethylene glycol, and the salt. The binding reagent that is used in the present method is also referred to herein as “precipitation reagent”. Details of the binding reagent are described below. Preferably, the binding conditions are exclusively established by the binding reagent and no further additives or reagents are added to establish the binding conditions for binding the nucleic acid molecules of different sizes to the solid phase which is also contacted with the binding mixture. Moreover, the binding reagent may be used to prepare the elution composition of step (c) by mixing a defined volume of the binding reagent with a defined volume of a dilution solution. This simplifies handling.

The term “poly(alkylene oxide) polymer” as used herein in particular refers to an oligomer or polymer of alkylene oxide units. Poly(alkylene oxide) polymers are known in low and high molecular weights. The molecular weight is usually a multitude of the molecular weight of its monomer(s) (e.g. 44 in case of ethylene oxide), and can range up to e.g. 50000. The molecular weight is indicated in Da. The poly(alkylene oxide) polymer may be linear or branched. A linear poly(alkylene oxide) polymer is preferred. The poly(alkylene oxide) polymer may be unsubstituted or substituted. Substituted poly(alkylene oxide) polymers, include e.g. alkylpoly(alkylene oxide) polymers, e.g. alkylpolyethylene glycols, but also poly(alkylene oxide) esters, poly(alkylene oxide) amines, poly(alkylene oxide) thiol compounds and others. The alkylene oxide unit may be selected from the group consisting of ethylene oxide and propylene oxide. Also co-polymers such as e.g. of ethylene oxide and propylene oxide are encompassed by the term a poly(alkylene oxide) polymer. Preferably, the poly(alkylene oxide) polymer is a poly(ethylene oxide) polymer or a poly(propylene oxide) polymer, more preferably it is a polyethylene glycol or a polypropylene glycol. Polyethylene glycol is particularly preferred because it is also commonly used in size selective DNA isolation methods as is also evidenced by the prior art discussed above. However, also other poly(ethylene oxide) polymers may be used such as substituted poly(ethylene oxide) polymers, e.g. alkyl poly(ethylene oxide) polymers such as alkylpolyethylene glycols. Polyethylene glycol is preferably unbranched and may be unsubstituted or substituted. Known substituted forms of polyethylene glycol include alkylpolyethylene glycols that are e.g. substituted at one or both ends with a C1-C5 alkyl group.

Preferably, unsubstituted polyethylene glycol is used as poly(alkylene oxide) polymer in the present invention. Such unsubstituted polyethylene glycol has the formula HO—(CH₂CH₂O)ₙ—H, wherein n depends on the molecular weight. All disclosures described in this application for the poly(alkylene oxide) polymer in general specifically apply and particularly refer to the preferred embodiment polyethylene glycol, in particular unsubstituted polyethylene glycol, even if not explicitly stated.

The poly(alkylene oxide) polymer can be used in various molecular weights as is demonstrated by the examples. According to one embodiment, the poly(alkylene oxide) polymer, which preferably is a polyethylene glycol, has a molecular weight that lies in a range of 2000 to 40000. The poly(alkylene oxide) polymer may have a molecular weight that lies in a range of 2500 to 35000 or 3000 to 30000, such as 4000 to 25000 or 5000 to 20000. As is supported by the examples, particular suitable ranges include 3000 to 25000, such as 6000 to 25000 and 8000 to 20000. Polyethylene glycol 3000, 8000 and 20000 was also used in the examples. Preferred is a molecular weight in the range of 6000 to 20000, such as in the range of 6000 to 16000, such as 8000. Such molecular weights are particularly suitable for polyethylene glycol. As disclosed herein, the molecular weight of the poly(alkylene oxide) polymer is indicated in Da.

The poly(alkylene oxide) polymer is present in the binding mixture in a concentration sufficient to precipitate nucleic acid molecules, such as extracellular nucleic acid molecules, having different sizes which then bind to the solid phase. The binding conditions are influenced and can be adjusted by the concentration of the poly(alkylene oxide) polymer in the binding mixture. Therefore, by varying the concentration of the poly(alkylene oxide) polymer in the binding mixture one may relocate and thus adjust the cut-off value for nucleic acid molecules that may bind to the solid phase. Increasing the concentration of the poly(alkylene oxide) polymer in the binding mixture lowers the mean length at which the nucleic acid molecules bind to the solid phase. Vice versa, a decrease in the concentration of the poly(alkylene oxide) polymer concentration increases the mean length at which nucleic acid molecules bind to the solid phase. Hence, the higher the concentration, the smaller the nucleic acid molecules that may bind to the solid phase. Such variation of the polymer concentration in the binding mixture can be achieved by preparing different binding reagents comprising the poly(alkylene oxide) polymer in a different concentration or by adding different volumes of the same binding reagent to the nucleic acid containing sample.

Binding conditions are provided in the binding mixture at which nucleic acid molecules of different sizes bind to the solid phase in step (a). Hence, small and large nucleic acid molecules are bound to the solid phase. The sizes of the bound nucleic acid molecules may range from a few nt up to ten thousand nt and more. As discussed in the background, nucleic acids comprised in cell-depleted or cell-free body fluids typically have a size from 130 nt to several thousand nt. DNA molecules having a size up to 600 nt usually correspond to the core extracellular DNA molecules, while larger DNA molecules often correspond to genomic DNA contaminations which may be present in the extracellular fraction of a body fluid. In one embodiment, the binding conditions provided in the binding mixture achieve the binding of DNA molecules having a length of 150 nt, 100 nt, 50 nt, 30 nt, or 20 nt to the solid phase. Important is that it is achieved that the small target DNA molecules to be enriched with the present method are bound to the solid phase. As discussed herein, this can be adjusted by adjusting the concentration of the poly(alkylene oxide) polymer (preferably a polyethylene glycol) in the binding mixture. When establishing binding conditions that allow to bind such small DNA molecules, larger DNA molecules will bind as well under these conditions. In one embodiment, predominantly nucleic acid of all sizes comprised in the nucleic acid sample bind to the solid phase in the binding step (a), except for e.g. single nucleotides or nucleic acids 30 or

According to one embodiment, the poly(alkylene oxide) polymer concentration in the binding mixture is at least 8% (w/v). The concentration may be at least 9%, at least 10%, at least 11% or at least 12% (w/v). The binding mixture may comprise the poly(alkylene oxide) polymer, which preferably is a polyethylene glycol, in a concentration that lies in a range of 8% to 30% (w/v). The concentration may e.g. lie in a range selected from 9% to 25% (w/v), 10% to 20%, 11% to 18% and 12% to 15% (w/v). Particularly suitable for binding nucleic acid molecules of different sizes from a cell-depleted or cell-free body fluid sample, wherein the bound nucleic acid molecules include extracellular DNA molecules of a length within a size range of 130-500 nt, is a poly(alkylene oxide) polymer (preferably polyethylene glycol) concentration in a range of 10% to 15% and 11% to 14% (w/v). All % with respect to the poly(alkylene oxide) polymer are indicated as (w/v). The required polymer concentration in the binding mixture in particular depends on the length of the smallest target nucleic acid molecules that shall be bound in step (a) and can be adapted based on the teachings provided herein.

The binding mixture comprises at least one salt. The salt promotes binding of the nucleic acid molecules to the solid phase. The salt can be a monovalent salt. As is demonstrated in the examples, a non-chaotropic salt is preferably used as salt. The salt may be an alkali metal salt, preferably a halide such as a chloride salt. It may be selected from sodium chloride, potassium chloride, lithium chloride and cesium chloride, e.g. selected from sodium chloride and potassium chloride. In one embodiment, the salt is sodium chloride. The use of a non-chaotropic alkali metal salt is preferred. According to one embodiment, the binding mixture does not comprise a chaotropic salt such as guanidinium salts, iodides, thiocyanates or perchlorates. Preferably, the binding mixture does not comprise other chaotropic salts of equal or stronger chaotropic nature either. In embodiments, the binding mixture does not comprise a C1-C8 alkanol in addition to the poly(alkylene oxide) polymer.

Suitable concentrations for the salt in the binding mixture are known from the prior art and can be determined by the skilled person based on the teachings provided herein. The binding mixture may comprise the salt in a concentration of 500 mM. The concentration may be 700 mM, 750 mM or 800 mM. Particularly suitable is a salt concentration of 1M, such as 1.25M and 1.5M. The salt may be present in the binding mixture in a concentration that lies in a range of 500 mM to 4M. Exemplary ranges include e.g. 750 mM to 3.5M, 1M to 3M, 1.25M to 2.5M and 1.5M to 2.25M. Particularly suitable for binding DNA molecules, e.g. from a cell-free or cell-depleted body fluid, is a salt concentration in the binding mixture that lies in a range of 1M to 2.5M, preferably 1.25M to 2.25M. The salt is preferably a monovalent salt, in particular a non-chaotropic alkali metal salt such as NaCl or KCl.

As disclosed herein, step (a) preferably comprises adding a binding reagent to the nucleic acid containing sample to prepare the binding mixture, wherein the binding reagent comprises the poly(alkylene oxide) polymer, preferably a polyethylene glycol, and the salt. The binding reagent is preferably liquid. It may be provided in form of a solution (which may comprise the solid phase, e.g. magnetic particles). A binding reagent that is added to the nucleic acid containing sample to prepare the binding mixture comprises the poly(alkylene oxide) polymer (preferably polyethylene glycol) and the salt in an amount that achieves the desired concentration in the binding mixture when contacting the intended volume of the nucleic acid containing sample with an appropriate volume of the binding reagent.

Suitable embodiments for the poly(alkylene oxide) polymer have been described above. The binding reagent may comprise the poly(alkylene oxide) polymer, which preferably is a polyethylene glycol, in a concentration of 10% to 50% (w/v). Suitable concentrations in the binding reagent may lie e.g. in a range selected from 11% to 45%, such as 12% to 40% and 15% to 35%. All % with respect to the polymer are indicated as (w/v). A molecular weight that lies in a range of 3000 to 30000, e.g. selected from 4000 to 25000, 5000 to 25000, 6000 to 20000 and 6000 to 16000, e.g. 8000, is particularly suitable. A volume of the binding reagent may be mixed with the nucleic acid containing sample to prepare a binding mixture that comprises the polymer, preferably a polyethylene glycol, in the above described concentrations.

Suitable embodiments for the salt have been described above. The binding reagent may comprise the salt, which preferably is an alkali metal salt, in a concentration that lies in the range of 0.5M to 5M. Suitable concentration ranges include but are not limited to 0.7M to 4.5M, 1M to 4.25M and 1.25M to 4M. Particularly preferred concentration ranges for the salt in the binding reagent are 1.5M to 3.75M and 1.75M to 3.5M. A volume of the binding reagent may be mixed with the nucleic acid containing sample to prepare a binding mixture that comprises the salt in the above described concentrations. It is referred to the above disclosure. As disclosed herein, it is preferred that the salt is a non-chaotropic salt.

According to one embodiment, a binding reagent is added in step (a) to prepare the binding mixture wherein the binding reagent comprises
a polyethylene glycol having a molecular weight that lies in the range of 3000 to 25000, preferably 4000 (or 5000) to 25000, such as 6000 to 20000 or 6000 to 16000; and
an alkali metal salt in a concentration that lies in the range 0.5M to 5M, preferably 0.7M to 4.5M, more preferably 1M to 4.25M, more preferably 1.5M to 3.75M or 1.75M to 3.5M.

According to one embodiment, a binding reagent is added in step (a) to prepare the binding mixture wherein the binding reagent comprises
a polyethylene glycol having a molecular weight that lies in a range of 5000 to 25000, e.g. selected from 6000 to 20000 and 6000 to 16000, such as 6000 to 10000; and
an alkali metal salt in a concentration that lies in the range of 1M to 4M, preferably 1.5M to 3.75M, more preferably 2M to 3.5M.

According to one embodiment, a binding reagent is added in step (a) to prepare the binding mixture wherein the binding reagent comprises
a polyethylene glycol having a molecular weight that lies in a range of 5000 to 25000, such as 6000 to 20000, in a concentration that lies in a range of 10% to 45% (w/v), e.g. selected from 11% to 40%, 12% to 35% and 15% to 30%; and
an alkali metal salt in a concentration that lies in a range of 1M to 4M, e.g. selected from 1.5M to 3.75M and 2M to 3.5M.

According to one embodiment, a binding reagent is added in step (a) to prepare the binding mixture wherein the binding reagent comprises
a polyethylene glycol having a molecular weight that lies in a range of 5000 to 25000, such as 6000 to 20000 or 6000 to 16000, in a concentration that lies in a range of 12% to 40%, preferably 15% to 35%;
an alkali metal salt, preferably selected from sodium chloride and potassium chloride, in a concentration selected from 1.5M to 3.5M and 2M to 3M.

Further binding reagents are also disclosed in the claims.

The binding reagent may comprise additional components. Exemplary components include but are not limited to a surfactant (e.g. a non-ionic surfactant) or a chelating agent. Chelating agents include, but are not limited to diethylenetriaminepentaacetic acid (DTPA), ethylenedinitrilotetraacetic acid (EDTA), ethylene glycol tetraacetic acid (EGTA) and N,N-bis(carboxymethyl)glycine (NTA) and furthermore, e.g. citrate or oxalate. EDTA is preferred. The binding reagent may also comprise a buffering agent.

According to one embodiment, the binding reagent does not comprise a chaotropic salt such as guanidinium salts, iodides, thiocyanates and perchlorates or other chaotropic salts of equal or stronger chaotropic nature. In embodiments, the binding reagent does not comprise a C1-C8 alkanol.

According to one embodiment, the binding reagent comprises the solid phase, which preferably is provided by particles, more preferably by magnetic particles. The magnetic particles, such as carboxylated magnetic particles, may be suspended in the liquid binding reagent. The binding reagent comprising the solid phase may then be contacted with the nucleic acid containing sample to prepare the binding mixture.

The binding conditions and hence length of the nucleic acid molecules that are bound to the solid phase can be and preferably are controlled/adjusted by the binding reagent that is added to the nucleic acid containing sample. As disclosed herein, binding conditions are established to ensure that the target nucleic acids, such as extracellular DNA molecules, comprised in cell-depleted or cell-free body fluids, are bound to the solid phase.

According to one embodiment, step (a) comprises adding X volume binding reagent to 1 volume nucleic acid containing sample, wherein X is a number that lies in a range from 0.2 to 3. X may be a number that lies in a range of 0.5 to 2.8, 0.7 to 2.7, 0.9 to 2.6, and 1 to 2.5. In one embodiment, X is ≥0.5, such as ≥0.6, ≥0.7, ≥0.8, ≥0.9, and preferably ≥1.0. According to one embodiment, X is ≤3.0, such as ≤2.9, ≤2.8, ≤2.7, ≤2.6, ≤2.5, ≤2.4, ≤2.3 or ≤2.2. Preferably X is a number that lies in a range of 1.0 to 2.5. According to an advantageous embodiment, X is a number that lies in arrange of 1.5 to 2.5, such as in the range of 1.6 to 2.0.

Contacting the binding reagent with the nucleic acid containing sample reduces the concentration of the ingredients contained in the binding reagent in the resulting binding mixture due to the dilution effect of the nucleic acid containing sample, which preferably is a liquid sample. By adding e.g. different amounts of the liquid binding reagent to the nucleic acid (e.g. DNA) containing sample, one may flexibly adjust the binding conditions.

As is described above, the binding mixture is preferably prepared by contacting the sample, which preferably is a cell-free or cell-depleted body fluid sample, with the binding reagent. In one embodiment, the binding reagent may have a pH value that lies in a range of 4 to 10. A suitable pH may lie e.g. in a range of 4.5 to 9.5 and 5 to 9. In one embodiment, the pH lies in a range of 7 to 8.5. Such pH is particularly suitable when using a solid phase comprising carboxyl groups. When using a solid phase comprising carboxyl groups, such as carboxylated magnetic particles, which is preferred, the pH can vary over a broad range. To maintain the pH, the binding reagent may comprise a buffering agent.

According to one embodiment, the binding conditions such as the concentration of the poly(alkylene oxide) polymer and the salt are established by contacting the nucleic acid containing sample with the binding reagent. Preferably, no further adjustments are made to establish the binding conditions in the binding mixture. Thus, preferably, the binding mixture is provided exclusively by contacting the binding reagent with the nucleic acid containing sample and the solid phase but no further buffers or other reagents are added to establish the binding conditions for binding the precipitated target nucleic acid molecules to the solid phase. This advantageously avoids handling and adjustment errors. Furthermore, as is described herein, the binding reagent may also advantageously be used to prepare the elution composition of step (c) by adding a dilution solution.

The nucleic acid containing sample may comprise DNA and/or RNA. Poly(alkylene oxide) polymer based size selective nucleic acid isolation is known for DNA and RNA. DNA is a more common application and preferred. Preferably, the nucleic acid containing sample is thus a DNA containing sample. It is preferably a liquid sample. The DNA containing sample may comprise single-stranded and/or double stranded DNA. The nucleic acids in the DNA containing sample comprise or consist of DNA molecules of different sizes (lengths). The nucleic acid containing sample can be of various origins, including but not limited to biological samples and artificial samples that were obtained during nucleic acid processing. The present method is particularly suitable for enriching target extracellular DNA molecules of a certain length (e.g. 600 nt or 500 nt) as separate fraction from a cell-free or cell-depleted body fluid which comprises extracellular nucleic acids of different lengths. The cell-free or cell-depleted body fluid may have been pretreated in advance, e.g. digested in order to liberate the extracellular nucleic acids that may be comprised in complexes as disclosed in the background section. Suitable samples are also described elsewhere herein.

The sizes (lengths), and also the cut-off values indicated herein, with reference to nucleotides “nt”, refer to the chain length of the nucleic acid molecules, which preferably are DNA molecules, and thus are used in order to describe the length of, respectively describe the cut-off value for single-stranded as well as double-stranded nucleic acid molecules. In double-stranded DNA molecules said nucleotides are paired. Hence, if the DNA is a double stranded molecule, the indications with respect to the size or length in “nt” refers to “bp”. Thus, if a double-stranded DNA molecule has a chain length, respectively size, of 100 nt, said double-stranded DNA molecule has a size of 100 bp. The same applies to the definition of the cut-off value.

Suitable solid phases can embody a variety of shapes and include, but are not limited to, particles, fibers, filter, a membrane or other supports on which a precipitated nucleic acid can bind. Suitable solid phases have sufficient surface area to permit efficient binding of nucleic acids. The use of particles, in particular magnetic particles, as solid phase is preferred.

A variety of surfaces may be utilized as is known in the prior art. The solid phase may comprise a surface which is coated with a functional group which reversibly bind the nucleic acid under the used binding conditions. The functional group may act as a bioaffinity adsorbent for polyalkylene glycol precipitated nucleic acid such as DNA. Suitable functionalized solid phases that can be used in order to bind precipitated nucleic acids in poly(alkylene oxide) polymer based size selective nucleic acid isolation methods are well-known in the art and therefore, do not need any detailed description. The functional groups may be of the same or different type and may be provided by ionic groups, e.g. ion exchange groups, preferably acidic groups. Acidic groups can be provided by carboxyl groups, sulfonate groups and silane ligands. Preferably, the solid phase comprises carboxyl groups as functional group. In one embodiment, the solid phase includes a surface coating that provides the functional groups such as carboxyl groups.

In a preferred embodiment, the solid phase provides a surface comprising carboxyl groups, also referred to herein as carboxylated surface. As is described herein and demonstrated in the examples, the use of a solid phase comprising carboxyl groups at the surface is particularly suitable and preferred in the context of the present invention. Unless indicated otherwise, all disclosures and embodiments described in this application for the use of a solid phase in general, specifically apply and particularly refer to this preferred embodiment wherein the solid phase comprises carboxyl groups, more preferably wherein the solid phase is provided by carboxylated magnetic particles.

In general, and by way of example, a carboxylated surface is a surface that is coated with or encompasses one or more carboxyl groups or moieties that are capable of reversibly and non-specifically associating with nucleic acid. Methods for coating a solid phase with functional groups, either directly or indirectly, are known in the art. For example, the functional groups (e.g. the carboxyl group COOH) can coat a solid phase during formation of the solid phase. In addition, the solid phases can be coated with functional groups by covalently coupling a functional group (one or more) to a COOH group (one or more) on the solid phase. A suitable moiety with a free carboxylic acid functional group is a succinic acid moiety in which one of the carboxylic acid groups is bonded to the amine of amino silanes through an amide bond and the second carboxylic acid is unbonded, resulting in a free carboxylic acid group attached or tethered to the surface of the solid phase.

According to one embodiment, particles are used as solid phase that may have the form of beads. The particles may have a size of about 0.02 to 25 μm, such as 0.1 to 15 μm, 0.125 to 12.5 μm, 0.15 to 10 μm and 0.2 to 7 μm. To ease the processing of the nucleic acid binding solid phase, preferably magnetic particles are used. Magnetic particles respond to a magnetic field. The magnetic particles may e.g. be ferrimagnetic, ferromagnetic, paramagnetic or superparamagnetic. Paramagnetic particles are particularly preferred. Paramagnetic particles can be efficiently separated from a solution using a magnet, but can be easily resuspended without magnetically induced aggregation occurring. Paramagnetic particles may comprise a magnetite rich core encapsulated by a polymer shell.

According to a preferred embodiment the solid phase is provided by carboxylated magnetic particles, wherein preferably, the magnetic particles are paramagnetic. Carboxylated magnetic particles are also commercially available, and include but are not limited to Sera-Mag Speed Beads (Sigma Aldrich, GE), Agencourt AMPure XP, QIAseq, M-Beads (MoBiTec).

The use of magnetic particles has advantages, because the magnetic particles including the bound nucleic acids can be processed easily by the aid of a magnetic field, e.g. by using a permanent magnet. This embodiment is e.g. compatible with established robotic systems capable of processing magnetic particles. Different robotic systems exist in the prior art that can be used in conjunction with the present invention to process the magnetic particles to which the target DNA molecules were bound. According to one embodiment, magnetic particles are collected at the bottom or the side of a reaction vessel and the remaining liquid sample is removed from the reaction vessel, leaving behind the collected magnetic particles to which the DNA molecules are bound. Removal of the remaining sample can occur by decantation or aspiration. Such systems are well known in the prior art and thus need no detailed description here. In an alternative system that is known for processing magnetic particles the magnet which is usually covered by a cover or envelope plunges into the reaction vessel to collect the magnetic particles. As respective systems are well-known in the prior art and are also commercially available (e.g. QIASYMPHONY®; QIAGEN), they do not need any detailed description here. In a further alternative system that is known for processing magnetic particles, the sample comprising the magnetic particles can be aspirated into a pipette tip and the magnetic particles can be collected in the pipette tip by applying a magnet e.g. to the side of the pipette tip. The remaining sample can then be released from the pipette tip while the collected magnet particles which carry the bound target DNA molecules remain due to the magnet in the pipette tip. The collected magnetic particles can then be processed further. Such systems are also well-known in the prior art and are also commercially available (e.g. BioRobot EZ1, QIAGEN) and thus, do not need any detailed description here.

According to one embodiment, the solid phase is comprised in a column. The term “column” as used herein in particular describes a container having at least two openings. Thereby, a solution and/or sample can pass through said column. The term “column” in particular does not imply any restrictions with respect to the shape of the container which can be e.g. round or angular and preferably is cylindrical. However, also other shapes can be used, in particular when using multi-columns. Said solid phase comprised in the column should allow the passage of a solution, respectively the binding mixture when applied to the column. This means that if e.g. a centrifuge force is applied to the column, a solution and/or the binding mixture is enabled to pass through the column in direction of the centrifuge force. When using a column based isolation procedure, the binding mixture is usually passed through the column, e.g. assisted by centrifugation or vacuum, and the nucleic acid molecules having a size above the cut-off value bind to the comprised solid phase during said passage.

Contacting the nucleic acid containing sample with the binding reagent to provide the binding mixture and binding of the target nucleic acid molecules to the solid phase may be performed simultaneously or sequentially. According to one embodiment, the nucleic acid containing sample is contacted with the binding reagent and the resulting binding mixture is then contacted with the solid phase. When using a particulate solid phase, the solid phase, the binding reagent and the nucleic acid containing sample can be added in any order. E.g. it is within the scope of the present invention to first provide the solid phase and the binding reagent (e.g. in form of a suspension) and then add the sample or to first provide the sample, the solid phase and then add the binding reagent. Preferably, the binding reagent is mixed with the nucleic acid containing sample to provide the binding mixture.

As discussed herein, the solid phase comprising a functional group is preferably provided by particles, more preferred magnetic particles, such as carboxylated magnetic particles. The particles may be comprised in the binding reagent. Binding can be supported by agitation, e.g. incubation on a shaker or other agitating instrument.

At the end of step (a), nucleic acid molecules of different sizes are bound to the solid phase.

### Step (b)

Separation step (b) is preferably performed in order to separate the bound nucleic acids from the sample remainders. The separated solid phase is then contacted with the elution composition of step (c) in order to selectively elute the target nucleic acid molecules having a length below the cut-off value. Alternatively, one may also dilute the binding mixture comprising the solid phase (e.g. magnetic particles) by adding a dilution solution to lower the concentration of the poly(alkylene oxide) polymer (and the salt) in the binding mixture to thereby prepare the elution composition of step (c).

However, step (b) is preferably performed and the nucleic acid molecules that are bound to the solid phase are separated from the remaining sample. Thereby, the bound nucleic acid molecules are separated from the sample remainders and thus impurities. Suitable separation methods are well known in the prior art and the appropriate separation technique also depends on the used solid phase. When using a particulate solid phase, which is preferred, the particles can be collected by sedimentation which can be assisted by centrifugation. Preferably, separation is performed with the aid of a magnet (magnetic separation) if magnetic particles are used. The supernatant can be separated off (e.g. decanted or aspirated) or the particles with the bound nucleic acids can be taken out of the liquid binding mixture. Suitable embodiments were described above in conjunction with the different formats of the solid phase and are well-known to the skilled person.

The bound nucleic acid molecules of different sizes may optionally be washed after separating the solid phase from the remaining sample. Thus, in one embodiment, at least one washing step is performed after separation in order to further purify the bound nucleic acid molecules of different sizes. A suitable washing solution removes impurities such as sample remainders but not the target nucleic acid molecules that are bound to the solid phase. According to one embodiment, no washing step is performed prior to step (c).

### Step (c)

Step (c) comprises contacting the solid phase with the bound nucleic acid molecules at least once with an elution composition comprising a poly(alkylene oxide) polymer and a salt to selectively elute the target nucleic acid molecules having a length below the cut-off value from the solid phase while larger nucleic acid molecules having a length above the cut-off value remain bound to the solid phase. In step (c), the concentration of the poly(alkylene oxide) polymer in the elution composition is lower than the concentration of the poly(alkylene oxide) polymer in the binding mixture of step (a). This achieves the size-selective elution of the target nucleic acid molecules.

The advantages of performing step (c) have been explained above. As discussed, step (c) efficiently and size selectively elutes target nucleic acid molecules having a desired length below the cut-off value from the solid phase. As is demonstrated in the examples, the elution of target nucleic acid molecules having a size below the cut-off value that is achieved in step (c) is very efficient. The elution efficiency for the target nucleic acid molecules can be adjusted by the stringency of the elution conditions used in step (c). As is demonstrated in the examples, a very high elution efficiency and thus enrichment of target nucleic acids having a length below a set cut-off value can be achieved with the present method. Depending on the concentration of the poly(alkylene oxide) polymer in elution composition (c), there might be some co-elution of longer nucleic acids. Depending on the further use, this is acceptable as an enrichment of the target nucleic acids having a length below the cut-off value can be nevertheless achieved and the elution efficiency for the target nucleic acids is high. If undesired, such co-elution can be substantially prevented by using a sufficiently high concentration of the poly(alkylene oxide) polymer in step (c). Suitable conditions can be chosen by the skilled person based on the disclosure provided herein and the intended further use of the eluted target nucleic acid molecules.

Several embodiments are feasible for contacting the solid phase with the bound nucleic acids with the elution composition, also depending on the type of solid phase used. When using a column, the elution composition may be added to the column so that it may flow through the column and elute the target nucleic acids.

As disclosed herein, the use of particles, in particular magnetic particles, such as carboxylated magnetic particles, is preferred. E.g. after separation, the solid phase may be transferred into the prepared elution composition, respectively the elution reagent (c). Alternatively, the elution composition, preferably in form of a single reagent, may be added to the solid phase. These embodiments are particularly feasible when using particles, such as magnetic particles, as solid phase. The elution composition may also be prepared by contacting a first reagent comprising the poly(alkylene oxide) polymer and a second reagent comprising the salt, either sequentially (in any order) or simultaneously, with the particles providing the solid phase, to achieve contacting the solid phase with the bound nucleic acid molecules with the elution composition that establishes the conditions of step (c). Preferably, the elution composition is provided by a single elution reagent, that comprises the poly(alkylene oxide) polymer and the salt, also referred to herein as reagent (c). As disclosed herein, the elution composition can be advantageously prepared from the binding reagent, e.g. by diluting the binding reagent with a suitable dilution reagent (e.g. a TE buffer as shown in the examples) in order to adjust the polymer concentration for the size-selective elution step (c).

The elution composition that is used in step (c), which preferably is provided by a single liquid reagent composition, also referred to herein as reagent (c), may have one or more of the characteristics of the binding reagent described above. As disclose herein, the liquid elution composition may be prepared from the binding reagent by dilution with a dilution reagent. Details regarding the type, molecular weight and concentration of the poly(alkylene oxide) polymer, which preferably is a polyethylene glycol, the type and concentration of the salt and potential further ingredients such as buffering agents, non-ionic detergents and chelating agents as well as components that are not comprised in embodiments were described above in conjunction with step (a) and it is referred to the above disclosure which also applies to the reagent composition/reagent used in step (c). Advantageously, the concentration of the poly(alkylene oxide) polymer is however lower in the elution composition of step (c) compared to the binding mixture of step (a) to efficiently promote the elution of the target nucleic acid molecules.

The elution composition/reagent (c) that is used in step (c) for selectively eluting the target nucleic acid molecules having a size below the cut-off value from the solid phase may comprise the poly(alkylene oxide) polymer, which preferably is a polyethylene glycol, in a concentration of at least 5% (w/v), such as at least 6%, at least 6.5 (w/v). According to one embodiment, the elution composition/reagent (c) comprises the poly(alkylene oxide) polymer, which preferably is a polyethylene glycol, in a concentration that lies in a range of 5% to 15% (w/v). Suitable concentration ranges include but are not limited to 5.5% to 12%, 6% to 11%, 6.25% to 10%, 6.5% to 9% and 6.5 to 8.5 (w/v). Suitable concentrations in the elution composition (c) can be chosen depending on the desired cut-off for eluting the target nucleic acids in step (c). As is shown in the examples, particularly suitable is a range of 6% or 6.5% to 8.5% in order to selectively elute extracellular DNA molecules. As disclosed herein, the concentration (w/v) of the polymer in the elution composition is lower compared to the concentration (w/v) used in the binding mixture.

The molecular weight of the poly(alkylene oxide) polymer, which preferably is a polyethylene glycol, may lie in a range of 2000 to 40000, such as 3000 to 30000 or 4000 to 30000. As shown in the examples, the molecular weight of the polymer that is used in step (c) for size-selective elution may influence the result. Polymers of higher molecular weights achieve more robust results in that non-target nucleic acids having a size above the cut-off value remain bound to the solid phase (e.g. particles) during the size selective elution, while the target nucleic acid could be efficiently eluted. Preferably, the molecular weight lies in a range of 4000 or 5000 to 25000, such as 6000 to 25000, 6000 to 20000 or 8000 to 20000. Another suitable range is 6000 to 16000, such as 6000 to 10000, e.g. 8000.

According to a preferred embodiment, polyethylene glycol is used as poly(alkylene oxide) polymer and the same type of polyethylene glycol is used in step (a) and step (c). The polyethylene glycol used in steps (a) and (c) accordingly may have the same molecular weight. According to a further embodiment, a polyethylene glycol of differing molecular weight is used in step (a) and step (c), wherein the molecular weight of the polymer that is used in step (c) is higher or lower than the molecular weight of the polyethylene glycol that is used in step (a). Preferably, the molecular weight of the polyethylene glycol that is used in step (c) is either the same or higher than the molecular weight of the polyethylene glycol that is used in step (a).

The elution composition that is contacted with the solid phase in step (c), respectively reagent (c), may comprise the salt in a concentration of 350 mM. The concentration in the elution composition, respectively reagent (c), may be 500 mM, 700 mM or 750 mM. Particularly suitable is a salt concentration of 700 mM and 750 mM for eluting extracellular DNA molecules as is demonstrated by the examples. The salt may be comprised in a concentration that lies in a range of 350 mM to 3.5M. Exemplary ranges include e.g. 500 mM to 3M, 600 mM to 2.5M, 700 mM to 2M, such as 725 mM to 1.5M and 750 mM to 1.25M. Particularly suitable is an elution composition, respectively a reagent (c), that comprises the salt in a concentration that lies in a range of 750 mM to 2M or 800 mM to 1.5M. The salt is preferably a monovalent salt, more preferably an alkali metal salt such as NaCl or KCl. The salt is preferably a non-chaotropic salt. The salt and suitable salt concentrations were also described in detail above. The elution composition, respectively reagent (c), preferably does not comprise a chaotropic salt. It is referred to the disclosure of the binding reagent which also applies here.

According to one embodiment, the solid phase is contacted in step (c) with an elution composition (c) which comprises
a polyethylene glycol having a molecular weight that lies in a range selected from 2000 to 40000, preferably in a range selected from 3000 to 35000, 4000 to 30000, 5000 to 25000, 6000 to 25000 and 6000 to 20000, such as 6000 to 16000; and
an alkali metal salt in a concentration that lies in a range of 350 mM to 3.5M, e.g. in a range selected from 500 mM to 3M, 600 mM to 2.5M, 700 mM to 2M, 750 mM to 1.5M and 800 mM to 1.25M. As disclosed herein, the salt is preferably a non-chaotropic salt such as NaCl or KCl.

Suitable and preferred molecular weights and concentrations for polyethylene glycol are also described above and can be used in elution composition (c). As disclosed herein, elution composition (c) may be prepared by diluting the binding reagent with a dilution reagent (e.g. a TE buffer).

According to one embodiment, the solid phase is contacted in step (c) with an elution composition (c) which comprises
a polyethylene glycol having a molecular weight that lies in a range of 3000 to 30000, preferably selected from 5000 to 25000, such as in a range of 6000 to 25000, e.g. 6000 to 20000, 6000 to 16000 or 8000 to 16000; and
an alkali metal salt in a concentration that lies in a range of 500 mM to 2.5M, preferably selected from 600 mM to 2M, 700 mM to 1.5M and 750 mM to 1.15M.

According to one embodiment, the solid phase is contacted in step (c) with an elution composition (c) which comprises
a polyethylene glycol having a molecular weight that lies in a range of 5000 to 25000, such as in a range of 6000 to 25000, 6000 to 20000 or 8000 to 20000, in a concentration that lies in a range of 5% to 12% (w/v), e.g. selected from 5.5% to 10%, 6% to 9% and 6.5% to 8.5%; and
an alkali metal salt in a concentration of 500 mM to 2.5M, e.g. selected from 600 mM to 2M, 700 mM to 1.5M and 750 mM to 1.15M.

According to one embodiment, the solid phase is contacted in step (c) with an elution composition (c) which comprises
a polyethylene glycol having a molecular weight that lies in a range of 5000 to 25000, such as in a range of 6000 to 25000, 6000 to 20000 or 8000 to 20000, in a concentration that lies in a range selected from 5.5% to 10% (w/v), such as 6% to 9% and 6.5% to 8.5% (w/v); and
an alkali metal salt in a concentration selected from 650 mM to 1.5M, 700 mM to 1.25M and 750 mM to 1.15M.

Elution compositions are also disclosed in the appended claims.

The concentration (w/v) of the poly(alkylene oxide) polymer in the elution composition of step (c) is lower than the concentration (w/v) of the poly(alkylene oxide) polymer in the binding mixture of step (a), to achieve an efficient elution of the target nucleic acids. The concentration of the salt in the elution composition of step (c) may be the same or is preferably lower than the concentration of the salt in the binding mixture of step (a). These features, especially in combination, provide very efficient elution conditions in step (c), thereby ensuring the efficient elution of the target nucleic acid molecules. The concentration of the poly(alkylene oxide) polymer and the salt can be lowered between step (a) and step (c) by the same ratio. This can be e.g. achieved by diluting the binding reagent comprising the poly(alkylene oxide) polymer and the salt with a dilution reagent, whereby the concentration of the polymer and the salt are lowered by the same ratio.

As is demonstrated in the examples, it is highly advantageous if the concentration (w/v) of the poly(alkylene oxide) polymer and, preferably also the concentration of the salt, in the elution composition of step (c) is lower compared to the binding mixture provided in step (a). This in particular applies if the molecular weight of the poly(alkylene oxide) polymer, which preferably is a polyethylene glycol, is the same during binding step (a) and size-selective elution step (c). As is demonstrated by examples the conditions of step (c) can be chosen such that target nucleic acids having a length below the cut-off value are efficiently depleted while the unwanted co-elution of larger non-target nucleic acid molecules is kept to a minimum.

As is shown in the examples, polymers of various molecular weights can be used in the size selective elution step (c). The concentration of the polymer in the elution composition/reagent (c) can be adjusted to ensure that the larger nucleic acids having a size above the cut-off value remain bound to the solid phase, while the target nucleic acids having a length below the cut-off value are selectively eluted. It is advantageous to use a poly(alkylene oxide) polymer, preferably a polyethylene glycol, that has a molecular weight of at least 5000, such as at least 6000 or at least 8000 (such as e.g. PEG 8000) at least in step (c) and preferably also in step (a), as the size-selective elution results are good and robust over a broader concentration range. As disclosed herein, the same type of poly(alkylene oxide) polymer may be used in step (a) and step (c), which may have the same molecular weight.

As disclosed herein, the binding conditions in step (a) are preferably established by adding a binding reagent that comprises the poly(alkylene oxide) polymer and the salt to the nucleic acid containing sample. The concentration (w/v) of the poly(alkylene oxide) polymer in the binding reagent that is added in step (a) is higher than the concentration (w/v) of the poly(alkylene oxide) polymer in the elution composition of step (c). As disclosed herein, the concentration (w/v) of the poly(alkylene oxide) polymer in the binding mixture of step (a) is higher than the concentration (w/v) of the poly(alkylene oxide) polymer in the elution composition of step (c). Furthermore, the concentration of the salt in the binding reagent that is added in step (a) is in one embodiment higher than the concentration of the salt in the elution composition of step (c). As disclosed herein, the concentration of the salt in the binding mixture of step (a) may be higher than the concentration of the salt in the elution composition of step (c). According to one embodiment, the concentration of the poly(alkylene oxide) polymer and the salt in the binding reagent that is added in step (a) and furthermore the binding mixture is higher than the concentration of the poly(alkylene oxide) polymer and the salt in the elution composition of step (c).

According to one embodiment, the elution composition of step (c) is provided by mixing a reagent comprising a poly(alkylene oxide) polymer and a salt (e.g. the binding reagent as disclosed herein), with a dilution reagent such as a dilution solution or dilution buffer. The reagent preferably comprises a poly(alkylene oxide) polymer and a salt as described before, in particular a polyethylene glycol and an alkali metal salt as described before. According to one embodiment, the dilution reagent comprises predominantly water. The dilution reagent may additionally comprise a buffering agent, which may be a buffering agent described in the present disclosure. According to a particular embodiment, the dilution reagent comprises Tris and EDTA (also referred to as TE buffer). The dilution reagent preferably does not comprise a poly(alkylene oxide) polymer and/or a salt. The reagent and the dilution reagent may be mixed by any method known in the art. Moreover, the reagent (e.g. the binding reagent used in step (a)) and the dilution reagent may be mixed at any ratio or factor suitable for forming an elution composition of step (c), as described herein. A volume of the reagent (e.g. the binding reagent as described herein) may be diluted with an appropriate volume of the dilution reagent in order to prepare an elution composition (c) as described herein. The ratio to be applied in particular depends on the concentration of the poly(alkylene oxide) polymer in the reagent that is diluted with the dilution solution and the concentration that is to be achieved in the elution composition of step (c).

According to one embodiment, X volume of the reagent (preferably the binding reagent used in step (a)) is mixed with 1 volume of the dilution reagent, such as a TE buffer. X may be selected from any number, for instance X may lie in the range of 0.1 to 2, such as 0.2 to 1.5, 0.3 to 1, 0.4 to 0.8 and 0.5 to 0.75. According to one embodiment, X is at least 0.3, such as at least 0.4, or at least 0.5. The binding reagent as disclosed may be used for preparing the elution composition for step (c) by mixing with a dilution reagent, such as a dilution solution. Details of the binding reagent are described elsewhere.

According to one embodiment, the solid phase with the bound nucleic acid is in step (c) only contacted with a single elution composition (c), but not with further reagents, such as further solutions. Therefore, according to one embodiment, the selective elution/binding conditions used in step (c) are exclusively established by reagent (c). As is demonstrated in the examples, reagent (c) may be advantageously provided, respectively be freshly prepared, by mixing e.g. the binding reagent that is used in step (a) with a dilution solution or buffer in order to provide/prepare reagent (c) that is then contacted with the separated solid phase. However, different contacting orders are also feasible.

As is described herein and demonstrated in the examples the use of a solid phase comprising carboxyl groups at the surface is particularly suitable and preferred in the context of the present invention. All disclosures described herein in the context of a solid phase in general, also specifically apply and refer to the use of a solid phase comprising carboxyl groups at the surface, such as carboxylated particles which preferably are magnetic carboxylated particles. The embodiments for the elution composition/reagent (c) disclosed above are particularly suitable for use in combination with a solid phase that comprises carboxyl groups, such as e.g. carboxylated magnetic particles.

For contacting, the solid phase with the bound nucleic acids may be incubated and moved, e.g. immersed, suspended or agitated, within the elution composition, respectively reagent (c). This is particularly feasible if using particles, preferably magnetic particles, for binding. The solid phase with the bound nucleic acids may be agitated, e.g. shaked, in the reagent to support the elution of the small non-target nucleic acid molecules.

Step (c) can furthermore be repeated. In this embodiment, the solid phase with the bound nucleic acid molecules having a length above the cut-off value is preferably separated from the elution composition of step (c) which comprises the eluted target nucleic acid molecules having a length below the cut-off value. The separated solid phase is then contacted again with an elution composition/reagent (c) comprising a poly(alkylene oxide) polymer and a salt to selectively elute further non-target nucleic acid molecules that may still be bound to the solid phase. The solid phase may be contacted with the same elution composition/reagent (c) that was used in the first size-selective elution step (c). Alternatively, a different elution composition/reagent (c) may be used, in which the concentration of the poly(alkylene oxide) polymer and/or the salt is lowered compared to the elution composition/reagent (c) that was used in the first size-selective elution step (c) to further promote elution of the target nucleic acid molecules having a length below the cut-off value that might have remained bound to the solid phase during the first elution step. In one embodiment, a dilution reagent may be added to further lower the concentration of the polymer and/or the salt in the elution composition after the first elution step in order to promote the further elution of still bound target nucleic acid molecules. According to one embodiment, step (c) is performed at least two times. As is demonstrated in the examples, repeating step (c) may further improve the results by even further eluting target nucleic acid molecules having a size below the desired cut-off from the solid phase. This may be advantageous if the nucleic acid containing sample comprises a high amount of target nucleic acid molecules having a length below the cut-off value. However, as is shown in the examples, an effective elution can already be achieved with a single elution step.

As disclosed herein, the concentration of the poly(alkylene oxide) polymer in the elution composition (c) influences the cut-off value and therefore influences which lengths of nucleic acid molecules remain bound to the solid phase, while smaller nucleic acid molecules are eluted into the elution composition.

According to one embodiment, a cut-off value is established in step (c) so that at least nucleic acid molecules having a length of 2000 nt remain bound to the solid phase.

According to one embodiment, a cut-off value is established in step (c) so that at least nucleic acid molecules having a length of 1500 nt remain bound to the solid phase.

According to one embodiment, a cut-off value is established in step (c) so that at least nucleic acid molecules having a length of 1000 nt remain bound to the solid phase.

According to one embodiment, a cut-off value is established in step (c) so that at least nucleic acid molecules having a length of 800 nt remain bound to the solid phase.

According to one embodiment, a cut-off value is established in step (c) so that at least nucleic acid molecules having a length of 600 nt remain bound to the solid phase.

According to one embodiment, a cut-off value is established in step (c) so that at least nucleic acid molecules having a length of 500 nt remain bound to the solid phase.

According to one embodiment, a cut-off value is established in step (c) by adjusting the concentration (w/v) of the poly(alkylene oxide) polymer and optionally the salt in the elution composition so that at least nucleic acid molecules having a length of 350 nt are eluted from the solid phase.

According to one embodiment, a cut-off value is established in step (c) by adjusting the concentration of the poly(alkylene oxide) polymer and optionally the salt in the elution composition so that at least nucleic acid molecules having a length of 500 nt or 600 nt are eluted from the solid phase.

According to one embodiment, a cut-off value is established in step (c) by adjusting the concentration of the poly(alkylene oxide) polymer and optionally the salt in the elution composition so that nucleic acid molecules having a length of 600 nt are eluted from the solid phase while larger nucleic acid molecules remain bound to the solid phase.

According to one embodiment, a cut-off value is established in step (c) by adjusting the concentration of the poly(alkylene oxide) polymer and optionally the salt in the elution composition so that nucleic acid molecules having a length of <500 nt are eluted from the solid phase while larger nucleic acid molecules remain bound to the solid phase.

According to one embodiment, the size selective elution performed in step (c) provides an eluted fraction of nucleic acid molecules wherein the majority of the nucleic acid molecules comprised in the eluted fraction have a length 2000 nt, preferably 1500 nt, 1000 nt, 800 nt, 700 nt, 600 nt, or 500 nt. The length of the eluted nucleic acids molecules fraction depends on the selected cut-off value.

### Step (d)

In step (d), the nucleic acid molecules having a length above the cut-off value that are still bound to the solid phase are separated from the selectively eluted target nucleic acid molecules. Suitable separation techniques are well known in the prior art and have been described above for step (b). Step (d) may be performed by the same or different means as in step (b). Details regarding the type of separation technique were described above in conjunction with step (b) and it is referred to the above disclosure which also applies to the separation step (d).

Thereby, an eluate is provided which comprises the eluted target nucleic acids having a length below the cut-off value.

### Step (e)

The eluted nucleic acid molecules can optionally be further purified in step (e). Step (e) may be performed after selectively eluting nucleic acid molecules having a length below the cut-off value in step (c) and separating the solid phase with the nucleic acid molecules having a length above the cut-off value bound thereto in step (d).

Basically any nucleic acid purification protocol can be used in step (e) to further purify the eluted target nucleic acid molecules, such as extracellular nucleic acid molecules. Examples for respective purification methods include but are not limited to extraction, solid-phase extraction, polysilicic acid-based purification, magnetic particle-based purification, phenol-chloroform extraction, anion-exchange chromatography (using anion-exchange surfaces), electrophoresis, precipitation and combinations thereof. It is also within the scope of the present invention to isolate specific nucleic acid molecules from the eluted target nucleic acid population, e.g. by using appropriate probes that enable a sequence specific binding and are coupled to a solid support. Also any other nucleic acid isolating technique known by the skilled person can be used. According to one embodiment, the target nucleic acid molecules are further purified from the provided eluate using at least one chaotropic agent and/or at least one alcohol, such as an C1-08, preferably C1-C4 alkanol. The nucleic acid molecules may isolated by binding them to a solid phase, preferably a solid phase comprising silicon. Suitable methods and kits are also commercially available such as the QIAamp® Circulating Nucleic Acid Kit (QIAGEN), the QIAamp MinElute Virus Spin or Vacuum Kit (QIAGEN), the Chemagic Circulating NA Kit (Chemagen), the NucleoSpin Plasma XS Kit (Macherey-Nagel), the Plasma/Serum Circulating DNA Purification Kit (Norgen Biotek), the Plasma/Serum Circulating RNA Purification Kit (Norgen Biotek), the High Pure Viral Nucleic Acid Large Volume Kit (Roche) and other commercially available kits suitable for purifying circulating nucleic acids. Here also automated protocols such as those running on the QIAsymphony system, the EZ1 insturments, the QlAcube (QIAGEN) or MagNApure system (Roche), m2000 sample prep systems (Abbott), EasyMag systems (bioMérieux) are available. Also any other available automated liquid-handling sample preparation system suitable for isolating the target nucleic acids can be used.

### Step (f)

According to one embodiment, the method further comprises a step (f) which comprises eluting nucleic acid molecules having a length above the cut-off value from the solid phase that was separated in step (d). Therefore, the larger nucleic acid molecules that were not eluted in step (c) and which accordingly were separated together with the solid phase may be obtained as separate fraction by eluting them from the solid phase. One or more elution steps may be performed in order to effectively release nucleic acid molecules having a length above the cut-off value from the separated solid phase. Optionally, the nucleic acid molecules eluted in step (f) may be further purified. Suitable methods were described above.

According to one embodiment, elution step (f) is performed by contacting the solid phase comprising the bound nucleic acid molecules having a length above the cut-off value with an elution solution. Here, basically any elution solution can be used which effects desorption of the bound nucleic acid from the solid phase in step (f). Common elution solutions known to effectively elute nucleic acids such as DNA include but are not limited to water (e.g. deionized water), elution buffers such as TE-buffer and low-salt solutions which have a salt content of 150 mM or less, e.g. 100 mM or less, preferably 75 mM or less, 50 mM or less, 25 mM or less, 20 mM or less, 15 mM or less, 10 mM or less or are salt-free. Commercially available elution solutions are e.g. buffers EB and AE (QIAGEN). The elution solution may e.g. comprise a buffering agent, in particular may comprise a biological buffer such as Tris, MOPS, HEPES, MES, BIS-TRIS, propane and others. The buffering agent may be present in a concentration of 150 mM or less, preferably 100 mM or less, more preferred 75 mM or less, 50 mM or less, 25 mM or less, 20 mM or less, 15 mM or less or 10 mM or less. According to one embodiment, the elution buffer has a pH value that is selected from pH 6 to pH 10, pH 7 to pH 9.5 and pH 7.5 to 9.0. Elution can be assisted by heating and/or shaking what is e.g. particularly feasible if a particulate solid phase is used for binding.

An elution solution should be used that does not interfere with the intended downstream application.

According to one embodiment, at least one wash step may be performed prior to elution step (f). For instance, at least one wash step can be performed after having separated the solid phase with the bound larger nucleic acid molecules from the eluted nucleic acid molecules in step (d). According to one embodiment, the used wash solution comprises at least one alcohol, preferably an alkanol. As alkanol, short chained branched or unbranched alcohols with preferably 1 to 5 carbon atoms can be used for washing, respectively can be used in the washing solution. Also mixtures of alcohols can be used. Suitable alcohols include but are not limited to methanol, ethanol, propanol, isopropanol and butanol. Preferably, isopropanol and/or ethanol are used in the washing solution. A further suitable washing solution which can be used alternatively or also in addition comprises an alcohol and a buffering agent. Suitable alcohols and buffering agents such as biological buffers are described above. Preferably, isopropanol or ethanol, most preferred ethanol is used in at least one washing step. Preferably, ethanol is used in a concentration of at least 50% (v/v), at least 60% (v/v) or at least 70% (v/v), preferably at least 80% (v/v). A further suitable washing solution which can be used alternatively or optionally also in addition to the washing solutions described above comprises an alkanol but no salt. This allows to wash away residual salts.

Residual alcohol that may be present after the washing step(s) in case an alcohol (e.g. alkanol) containing washing solution was used can be removed e.g. by air drying (e.g. suitable when working with a particulate solid phase) or by an additional separation step (e.g. centrifugation, magnetic separation, sedimentation, etc.). Respective methods and procedures are well-known in the prior art and thus, do not need any further description here.

### FURTHER EMBODIMENTS

Non-limiting preferred embodiments and applications of the method according to the present invention will be described further in the following. As disclosed herein, the size selective nucleic acid separation method according to the present invention is in particular suitable for enriching nucleic acid molecules having desired size ranges from a mixed population of nucleic acid molecules having different lengths/sizes. The method is in particular suitable for separation extracellular nucleic acids by their size and thus isolating fractions of nucleic acid molecules having different size ranges from a nucleic acid containing sample, wherein the same is preferably a cell-free or cell-depleted body fluid sample.

According to one embodiment, the method is for enriching target extracellular DNA molecules having a length below a cut-off value from a cell-depleted or cell-free body fluid sample, wherein the method comprises
(a) preparing a binding mixture comprising
   the cell-depleted or cell-free body fluid sample, which optionally is a digested sample,
   a polyethylene glycol in a concentration of at least 10%, preferably in a range of 12% to 25%, wherein the polyethylene glycol has a molecular weight that lies in a range of 3000 to 30000, preferably in a range of 5000 to 25000, and
   the salt in a concentration of 750 mM, preferably at least 1M, wherein the salt is an alkali metal salt, preferably a non-chaotropic alkali metal salt, more preferably selected from sodium chloride and potassium chloride,
   and binding nucleic acid molecules of different sizes to the solid phase, wherein the solid phase is provided by carboxylated magnetic particles and the bound nucleic acids include the target extracellular DNA molecules;
(b) separating the solid phase with the bound nucleic acid molecules from the remaining sample and optionally washing the bound nucleic acid molecules;
(c) contacting the solid phase with the bound nucleic acid molecules at least once with an elution composition to selectively elute the target extracellular DNA having a size below the set cut-off value from the solid phase while DNA molecules having a size above the cut-off value remain bound to the solid phase, wherein the elution composition comprises
   a polyethylene glycol having a molecular weight that lies in a range of 3000 to 30000, preferably in a range of 5000 to 25000, in a concentration that lies in a range from 5% to 10%, preferably 6% to 9% or 6.5% to 8.5% (w/v);
   the salt in a concentration of at least 500 mM, preferably 750 mM wherein the salt is an alkali metal salt, preferably a non-chaotropic alkali metal salt, more preferably selected from sodium chloride and potassium chloride, and
   wherein the concentration (w/v) of the polyethylene glycol in the elution composition is lower than the concentration (w/v) of the polyethylene glycol in the binding mixture of (a); and
(d) separating the solid phase with the still bound larger nucleic acid molecules from the eluted extracellular DNA molecules.

The method can be used for enriching extracellular DNA molecules having a length below a cut-off value of 1000 nt, 800 nt or 600 nt from larger DNA molecules comprised in the cell-free or cell-depleted body fluid sample. According to one embodiment, the eluate that is provided as result of performing steps (c) and (d) comprises predominantly extracellular DNA molecules having a length 1000 nt, 800 nt or preferably 600 nt. The method optionally further comprise step (e) and/or step (f):
(e) further purifying the eluted target extracellular DNA molecules;
(f) eluting nucleic acid molecules having a length above the cut-off value from the solid phase that was separated in step (d).

Step (f) allows to obtain also longer DNA molecules (such as HMW extracellular DNA) as separate fraction for analysis.

According to one embodiment, the method is for enriching target extracellular DNA molecules having a length below a cut-off value from a cell-depleted or cell-free body fluid sample, wherein the method comprises
(a) contacting a binding reagent that comprises polyethylene glycol as poly(alkylene oxide) polymer and a salt with a cell-free or cell-depleted body fluid sample, which optionally is a digested sample, thereby preparing a binding mixture comprising
   the cell-depleted or cell-free body fluid sample, which optionally is a digested sample,
   a polyethylene glycol in a concentration that lies in a range of 12% to 25%, wherein the polyethylene glycol has a molecular weight that lies in a range of 5000 to 25000, and
   the salt in a concentration of wherein the salt is an alkali metal salt, preferably a non-chaotropic alkali metal salt, more preferably selected from sodium chloride and potassium chloride, and binding nucleic acid molecules of different sizes to the solid phase, wherein the solid phase is provided by carboxylated magnetic particles and the bound nucleic acids include the target extracellular DNA molecules;
(b) separating the solid phase with the bound nucleic acid molecules from the remaining sample and optionally washing the bound nucleic acid molecules;
(c) contacting the solid phase with the bound nucleic acid molecules at least once with an elution composition to selectively elute the target extracellular DNA having a size below the set cut-off value from the solid phase while DNA molecules having a size above the cut-off value remain bound to the solid phase, wherein the elution composition comprises
   a polyethylene glycol having a molecular weight that lies in a range of 3000 to 30000, preferably in a range of 5000 to 25000, in a concentration that lies in a range from 5% to 10%, preferably 6% to 9% or 6.5% to 8.5% (w/v);
   the salt in a concentration of at least 500 mM, preferably 750 mM wherein the salt is an alkali metal salt, preferably a non-chaotropic alkali metal salt, more preferably selected from sodium chloride and potassium chloride, and
   wherein the concentration (w/v) of the polyethylene glycol in the elution composition is lower than the concentration (w/v) of the polyethylene glycol in the binding mixture of (a) and wherein the concentration of the salt in the elution composition is lower than the concentration of the salt in the binding mixture of (a), and wherein the elution composition is provided by diluting the binding reagent used in step (a) with a dilution solution; and
(d) separating the solid phase with the still bound larger nucleic acid molecules from the eluted extracellular DNA molecules.

The method can be used for enriching extracellular DNA molecules having a length below a cut-off value of 1000 nt, 800 nt or 600 nt from larger DNA molecules comprised in the cell-free or cell-depleted body fluid sample. According to one embodiment, the eluate that is provided as result of performing steps (c) and (d) comprises predominantly extracellular DNA molecules having a length 1000 nt, 800 nt or preferably 600 nt. The method optionally further comprise step (e) and/or step (f):
(e) further purifying the eluted target extracellular DNA molecules;
(f) eluting nucleic acid molecules having a length above the cut-off value from the solid phase that was separated in step (d).

### Sample

According to a preferred embodiment the nucleic acid containing sample is a cell-free or cell depleted biological sample which comprises extracellular nucleic acids. A biological sample is obtained from a biological source. The sample is not an artificial sample with synthetically produced nucleic acids but is obtained from a biological source. According to a preferred embodiment, the biological sample comprising the extracellular nucleic acids is a cell-free or cell-depleted body fluid sample. The body fluid may be naturally cell-free or a respective cell-free or cell-depleted sample can be obtained e.g. from a cell-containing body fluid sample by using appropriate technologies to remove cells. A typical example is blood plasma or blood serum which can be obtained from whole blood. A further example is urine, from which cells can be removed. Separating the cells from the cell-containing body fluid provides a cell-free, respectively cell-depleted body fluid sample which comprises the extracellular nucleic acids. Thus, according to one embodiment, cells are removed from a cell-containing sample such as a cell-containing body fluid, to provide the cell-free or cell-depleted sample which comprises extracellular nucleic acids. This cell removal step is optional and e.g. may be obsolete if samples are processed (respectively are obtained for processing) which merely comprise minor amounts of residual cells such as e.g. plasma or serum. However, in order to improve the results it is preferred that also respective remaining cells (or potentially remaining cells) are removed. Depending on the sample type, cells, including residual cells, can be separated and removed e.g. by centrifugation, preferably high speed centrifugation, or by using means other than centrifugation, such as e.g. filtration, sedimentation or binding to surfaces on (optionally magnetic) particles if a centrifugation step is to be avoided. Respective cell removal steps can also be easily included into an automated sample preparation protocol. Respectively removed cells may also be processed further e.g. in order to analyse the intracellular nucleic acids. The cells can e.g. be stored and/or biomolecules such as e.g. nucleic acids or proteins can be isolated from the removed cells.

According to one embodiment, the nucleic acid containing sample is selected from the group consisting of body fluids, body secretions, nasal secretions, vaginal secretions, wound secretions and excretions. It may be preferably selected from blood, plasma, serum, urine, saliva, lymphatic fluid, liquor, ascites, milk, bronchial lavage, sputum, amniotic fluid, semen/seminal fluid. In a preferred embodiment, the nucleic acid containing sample is selected from plasma, serum, urine, saliva and/or liquor. It may be selected from plasma or urine. In order to enrich extracellular nucleic acids as target nucleic acid molecules, it is preferred to deplete cells from the sample prior to enriching extracellular nucleic acid molecules having a length below the cut-off value from the obtained cell-free or cell-depleted sample using the method of the present disclosure.

As discussed in the background section, extracellular nucleic acid molecules, in particular extracellular DNA typically has different sizes ranging from about 100 nt up to 10,000 nt and more. It is of interest to enrich extracellular nucleic acid species according to their size (e.g. comprising extracellular DNA having a length of ≤600 nt) in order to analyse them as separate fraction. This can be advantageously achieved by the method according to the present disclosure which allows to remove higher molecular weight nucleic acids, such as residual genomic DNA can be present in the cell-free or cell depleted sample. In order to analyse the extracellular nucleic acids separately from those other nucleic acid species, the binding and/or selective elution conditions of method step (a) and (d) can be adjusted to the desired cut-off value.

According to one embodiment, the sample is digested, e.g. lysed, prior to binding step (a). An according digestion step can support the release of the target nucleic acids to be enriched which therefore, can be better bound to the solid phase. E.g. as discussed in the background, extracellular nucleic acids are often comprised in proteolipid complexes, vesicles or are associated with proteins. Thus, a digestion step may also be performed when processing a cell-free or cell-depleted body fluid sample in order to make the extracellular nucleic acids better available for enrichment. Suitable digestion methods are known in the art and include e.g. the use of a protease, a surfactant, a base and/or a denaturing agent. Accordingly, a digested nucleic acid containing sample may be processed in step (a).

### Cut-Off Value

The cut-off value defines a size or size range at which the majority of the nucleic acid molecules bind or remain bound to the solid phase if they have a size above the cut-off value or do not bind/are eluted if they have a size below the cut-off value. The expression that “nucleic acid molecules having a length above the cut-off value remain bound to the solid phase” and similar expressions used herein, in particular specify that nucleic acid molecules having a size at the cut-off value or above remain bound to the solid phase. I.e. if the cut-off value for nucleic acid molecules is described as being 600 nt, this means that nucleic acid molecules having a length of 600 nt or longer predominantly remain bound to the solid phase. Thus, the cut-off value in particular defines here the length/size of the smaller nucleic acid molecules that substantially do not bind or are eluted under the respective binding/elution conditions to the solid phase. However, at this point, respectively this cut-off value, there is not necessarily a quantitative recovery of the nucleic acids but the percentage of captured nucleic acid molecules increases with increasing length/size of the nucleic acid molecules. According to one embodiment, the cut-off value corresponds to the point where the curve of an electropherogram for the nucleic acid molecules having a size above the cut-off value, e.g. HMW species, meets the x-axis.

Performing the size selective elution step (c) has several advantages. It allows to size-selectively elute extracellular DNA molecules of a certain size or size range as target nucleic acids. Elution conditions can be used, wherein high molecular weight nucleic acids such as for example genomic DNA or other longer intracellular DNA molecules, are not recovered but remain bound to the solid phase. The method according to the present invention thereby allows to eliminate respective intracellular nucleic acid contaminations such as e.g. genomic DNA in the enriched target nucleic acids. According to one embodiment, size selective elution conditions are used in step (c), so that predominantly nucleic acids having a size 2,000 nt, preferably 1,500 nt, 1,000 nt, 800 nt, 700 nt or more preferably 600 nt such as e.g. <500 nt are size-selectively eluted and thus present in the obtained eluate. Such cut-off values are particularly suitable for enriching extracellular DNA molecules, while depleting high molecular weight species that correspond to genomic DNA. According to one embodiment, size selective elution performed in step (c) provides an eluted fraction of nucleic acid molecules wherein the comprised nucleic acid molecules have a length up to 2,000 nt, up to 1,500 nt, up to 1,000 nt, up to 800 nt, up to 700 nt or up to 600 nt, depending on the selected cut-off value. According to one embodiment, the eluted fraction obtained after step (c) comprises fraction comprises mono-, di- and/or trinucleosomal ccfDNA.

If not all small nucleic acids of interest bind to the solid phase in step (a), they are still comprised in the supernatant that is obtained after the binding step (a) and separation step (b). These remaining nucleic acids predominantly consist of small nucleic acid molecules having a length below the cut-off value. This is because the larger DNA molecules effectively bind to the solid phase under the binding conditions and are thus removed together with the solid phase. If all small nucleic acids are to be recovered from the sample or should be recovered with a greater yield, the supernatant of the binding step that is obtained after steps (a) and (b) can be used as additional source for size-selectively enriched small target nucleic acids having a length below the cut-off value. The supernatant obtained from the binding step may be further purified to recover the comprised small target nucleic acids. The supernatant of the binding step may be e.g. purified along with the supernatant(s) obtained from the size-selective elution step(s) (c), which comprise the size-selectively eluted target nucleic acids having a length below the cut-off value. This way it is possible to recover the entire smaller DNA-fraction from ccfDNA as enriched fraction.

Fractionation of Nucleic Acids According to their Size

According to one embodiment, at least two cycles of steps (c) and (d) are performed for providing different nucleic acid fractions comprising nucleic acid molecules of different sizes, wherein the cut-off value of a preceding cycle is lower compared to the cut-off value of a subsequent cycle and wherein the nucleic acid molecules eluted in a preceding cycle are smaller than the nucleic acid molecules eluted in a subsequent cycle. Hence, the nucleic acid molecules eluted in a preceding cycle are smaller than the nucleic acid molecules eluted in a subsequent cycle.

Thus, in the subsequent elution step(s) conditions are applied which allow to elute longer nucleic acids (i.e. nucleic acids having a length below a cut-off value, which is, however, higher than the cut-off value of the preceding selective elution step). According to one embodiment, the cut-off value of the first cycle lies in the range of 200 nt to 600 nt and the cut-off value of a subsequent cycle lies in the range of 500 nt to 2000 nt. The concentration of the poly(alkylene oxide) polymer and preferably the salt of the elution composition used in a preceding cycle is higher than the concentration of the poly(alkylene oxide) polymer and preferably the salt of the elution composition that is used in a subsequent cycle. Therefore, because the method according to the present invention provides the possibility to control the size of the eluted nucleic acids by varying the elution conditions, it is very flexible.

### Automation

The method is advantageously suitable for automation. Here, it is favourable to use magnetic particles for providing the solid phase. According to one embodiment, at least steps (b) to (e) are performed on a sample processing system. The sample processing system has one or more of the following characteristics:
a) it is an automated system;
b) it does not comprise a pipetting unit;
c) it comprises at least two magnets for processing magnetic particles.

According to one embodiment, an automated system is used that does not require a pipetting unit. A corresponding system is e.g. disclosed in US2016/0202157, herein incorporated by reference, and is commercially available as Extractman®.

Suitable and preferred embodiments for the individual steps, in particular steps (a) and (c) as well as suitable and preferred binding reagents and reagents (c) were described above and can be used in the methods described herein as further embodiments.

Method for Isolating Extracellular DNA Molecules from a Cell-Free or Cell-Depleted Body Fluid Sample

According to a second aspect, a method is provided for enriching target extracellular DNA molecules having a length below a cut-off value from a cell-depleted or cell-free body fluid sample, comprising enriching target extracellular DNA molecules from the sample using the method according to the first aspect. Details of said method as well as suitable and preferred embodiments for a cell-free or cell-depleted body fluid sample have been described above and it is referred to the above disclosure.

According to one embodiment, the method comprises
(a) contacting a binding reagent that comprises polyethylene glycol as poly(alkylene oxide) polymer and a salt with a cell-free or cell-depleted body fluid sample, which optionally is a digested sample, thereby preparing a binding mixture comprising
   the sample,
   polyethylene glycol in a concentration that lies in a range of 10% to 25% (w/v), preferably 12% to 20% (w/v), wherein the polyethylene glycol has a molecular weight that lies in a range of 3000 to 30000, preferably in a range of 5000 to 25000, and
   the salt in a concentration of 1M, wherein the salt is an alkali metal salt, preferably a non-chaotropic alkali metal salt chloride, more preferably selected from sodium chloride and potassium chloride,
   and binding nucleic acid molecules of different sizes to the solid phase which comprises carboxyl groups as functional group, wherein the solid phase is preferably provided by carboxylated magnetic particles, and the bound nucleic acids include the target extracellular DNA molecules;
(b) separating the solid phase with the bound nucleic acid molecules from the remaining sample and optionally washing the bound nucleic acid molecules;
(c) contacting the solid phase with the bound nucleic acid molecules at least once with an elution composition to selectively elute the target extracellular DNA molecules having a size below the set cut-off value from the solid phase while DNA molecules having a size above the cut-off value remain bound to the solid phase, wherein the elution composition comprises
   a polyethylene glycol having a molecular weight that lies in a range of 3000 to 30000, preferably in a range of 5000 to 25000, in a concentration that lies in a range from 5% to 10%, preferably 6% to 9%, more preferably 6.5% to 8.5% (w/v);
   the salt in a concentration of at least 500 mM, preferably 750 mM wherein the salt is an alkali metal salt, preferably a non-chaotropic alkali metal salt chloride, more preferably selected from sodium chloride and potassium chloride, and
   wherein the concentration (w/v) of the polyethylene glycol in the elution composition is lower than the concentration (w/v) of the polyethylene glycol in the binding mixture of (a) and wherein the concentration of the salt in the elution composition is lower than the concentration of the salt in the binding mixture of (a), optionally wherein the elution composition is provided by diluting the binding reagent used in step (a) with a dilution solution; and
(d) separating the solid phase with the bound larger DNA molecules from the eluted target extracellular DNA molecules.

The method can be used for enriching extracellular DNA molecules having a length below a cut-off value of 1000 nt, 800 nt or 600 nt from larger DNA molecules comprised in the cell-free or cell-depleted body fluid sample. According to one embodiment, the eluate that is provided as result of performing steps (c) and (d) comprises predominantly extracellular DNA molecules having a length 1000 nt, 800 nt or preferably 600 nt.

The method optionally further comprise step (e) and/or step (f):
(e) further purifying the eluted target extracellular DNA molecules;
(f) eluting nucleic acid molecules having a length above the cut-off value from the solid phase that was separated in step (d).

### Applications

The methods according to the first and second aspect provide size-selectively enriched target nucleic acids having a size below the cut-off value. The enriched nucleic acids having a size below the cut-off value as well as the nucleic acids having a size above the cut-off value, if recovered, may be directly analysed and/or further processed using suitable assay and/or analytical methods. If a respective direct use is intended, it is preferred that the eluted nucleic acid molecules are further purified after having been (selectively) eluted, in particular if downstream assays are used that are sensitive to impurities or wherein a different composition than the one provided in the elution composition is required. Methods for further purification have been described above.

The nucleic acid molecules, which are preferably extracellular nucleic acid molecules, such as extracellular DNA, can be identified, quantified, modified, contacted with at least one enzyme, amplified, reverse transcribed, cloned, sequenced, contacted with a probe and/or be detected. Respective methods are well-known in the prior art and are commonly applied in the medical, diagnostic and/or prognostic field in order to analyse extracellular nucleic acids. Thus, after extracellular nucleic acids were isolated, optionally as part of total nucleic acid, total RNA and/or total DNA (see above), they can be analysed to identify the presence, absence or severity of a disease state including but not being limited to a multitude of neoplastic diseases, in particular premalignancies and malignancies such as different forms of cancers. E.g. the isolated extracellular nucleic acids can be analysed in order to detect diagnostic and/or prognostic markers (e.g., fetal- or tumor-derived extracellular nucleic acids) in many fields of application, including but not limited to non-invasive prenatal genetic testing respectively screening, disease screening, oncology, cancer screening, early stage cancer screening, cancer therapy monitoring, genetic testing (genotyping), infectious disease testing, pathogen testing, injury diagnostics, trauma diagnostics, transplantation medicine or many other diseases and, hence, are of diagnostic and/or prognostic relevance. According to one embodiment, the isolated extracellular nucleic acids are analyzed to identify and/or characterize a disease infection or a fetal characteristic. Thus, as discussed above, the isolation method described herein may further comprise a step c) of nucleic acid analysis and/or processing. The analysis/further processing of the nucleic acids can be performed using any nucleic acid analysis/processing method including, but not limited to amplification technologies, polymerase chain reaction (PCR), isothermal amplification, reverse transcription polymerase chain reaction (RT-PCR), quantitative real time polymerase chain reaction (Q-PCR), digital PCR, gel electrophoresis, capillary electrophoresis, mass spectrometry, fluorescence detection, ultraviolet spectrometry, hybridization assays, DNA or RNA sequencing, restriction analysis, reverse transcription, NASBA, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof. Respective technologies are well-known to the skilled person and thus, do not need further description here.

According to one embodiment, the enriched target nucleic acid molecules, such as extracellular nucleic acids, are analysed to identify, detect, screen for, monitor or exclude a disease, an infection and/or at least one fetal characteristic.

### Kit

Furthermore a kit is provided for the size selective enrichment of nucleic acid molecules, preferably extracellular DNA molecules, having a length below a cut-off value from a nucleic acid containing sample, comprising
(a) a binding reagent comprising at least one poly(alkylene oxide) polymer and at least one salt;
(b) magnetic particles for binding target nucleic acid molecules in the presence of the binding reagent (a); and
(c) an elution reagent comprising at least one poly(alkylene oxide) polymer and at least one salt and/or a dilution reagent for preparing the reagent (c) by combining the dilution reagent with the binding reagent;
(d) optionally at least one washing solution; and
(e) optionally an elution solution,
wherein the concentration of the poly(alkylene oxide) polymer in the binding reagent (a) is higher than the concentration of the poly(alkylene oxide) polymer in the reagent (c).

Such kit can be used e.g. in the method according to the first and second aspect. Details regarding the binding reagent, in particular suitable and preferred binding reagent components, binding reagent component concentrations, as well as details regarding the solid phase, elution reagent (c), and optional washing and elution solutions were described in detail above in conjunction with the method according to the first aspect. It is referred to the above disclosure which also applies here. Non-limiting selected embodiments are again described subsequently.

Suitable and preferred types and concentrations for the poly(alkylene oxide) polymer in the binding reagent were described above and it is referred to the above disclosure. Preferably, the poly(alkylene oxide) polymer is a poly(ethylene oxide) polymer, preferably a polyethylene glycol, more preferably unsubstituted polyethylene glycol.

Suitable and preferred types and concentrations for the salt in the binding reagent, which preferably is an alkali metal salt, preferably an alkali metal halide, such as a chloride, have been described above and it is referred to the above disclosure. The salt is preferably selected from sodium chloride, potassium chloride, lithium chloride and cesium chloride, more preferably the salt is sodium chloride. The salt is preferably a non-chaotropic salt.

Specific examples for binding reagents are furthermore disclosed in conjunction with the method according to the present invention and also in the claims and these binding reagents can be included in the kit according to the third aspect.

Details regarding elution reagent (c) which comprises at least one poly(alkylene oxide) polymer and at least one salt and may be used in method step (c) to remove nucleic acid molecules having a size below the cut-off were described in detail above in conjunction with the method according to the present invention and also in the claims and it is referred to the above disclosure. Any one of these elution reagents/elution compositions (c) can be included in the kit according to the third aspect. As described above, elution reagent/elution composition (c) may also be created by adding a dilution reagent (e.g. TE buffer) to the binding reagent (a) to thereby freshly prepare elution reagent/elution composition (c). The dilution reagent may be mixed with an appropriate volume of the binding reagent to prepare elution reagent/elution composition (c). The kit may also comprise the dilution reagent as kit component. Details of the dilution reagent, e.g. a dilution solution, were described above and it is referred to this disclosure.

Suitable and preferred embodiments of the solid phase were also described in conjunction with the method according to the first aspect and it is referred to the above disclosure. As described above, the solid phase preferably provides a carboxylated surface. Particularly preferred is the use of carboxylated magnetic particles. In one embodiment, the solid phase is comprised in the binding reagent (a).

Furthermore, the kit may comprise instructions and/or information for use. E.g. the kit may comprise instructions and/or information regarding the cut-off value that is achieved when mixing a certain volume of the binding buffer with a certain volume of the nucleic acid containing sample and/or the cut-off value(s) that are achieved if the nucleic acid containing sample is mixed in a certain ratio with the binding reagent. If two or more binding reagents are comprised in the kit that differ in the concentration of the poly(alkylene oxide) polymer, the kit may provide information which cut-off value is achieved when using a certain binding buffer comprised in the kit. Thus, the present invention also provides a kit which allows the flexible adjustment of the cut-off value e.g. by mixing a certain volume of the binding buffer and a certain volume of the nucleic acid containing sample.

A respective kit can be in particular used in the method according to the first or second aspect.

### Use

According to a fourth aspect, the present disclosure is directed to the use of a kit according to the third aspect in a method according to the first or second aspect. Specifically, the present disclosure provides a kit as defined in any one of claims 25 to 26 in a method as defined in any one of claims 1 to 24. Details regarding these aspects are described above and in the claims it is referred to the respective disclosure.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

The term “solution” as used herein in particular refers to a liquid composition, preferably an aqueous composition. It may be a homogenous mixture of only one phase but it is also within the scope of the present invention that a solution comprises solid constituents such as e.g. precipitates.

As used in the subject specification and claims, the singular forms “a”, “an” and “the” include plural aspects unless the context clearly dictates otherwise. Thus, for example, reference to “a poly(alkylene oxide) polymer” includes a single type of poly(alkylene oxide) polymer, as well as two or more poly(alkylene oxide) polymers. Likewise, reference to “a salt”, “a buffering agent” and the like includes single entities and combinations of two or more of such entities. Reference to “the disclosure” and “the invention” and the like includes single or multiple aspects taught herein; and so forth. Aspects taught herein are encompassed by the term “invention”.

The solid phase is not considered when determining the concentrations of the components, such as the poly(alkylene oxide) polymer or the salt in the binding mixture.

According to one embodiment, subject matter described herein as comprising certain steps in the case of methods or as comprising certain ingredients in the case of compositions, solutions and/or buffers refers to subject matter consisting of the respective steps or ingredients. It is preferred to select and combine preferred embodiments described herein and the specific subject-matter arising from a respective combination of preferred embodiments also belongs to the present disclosure.

### EXAMPLES

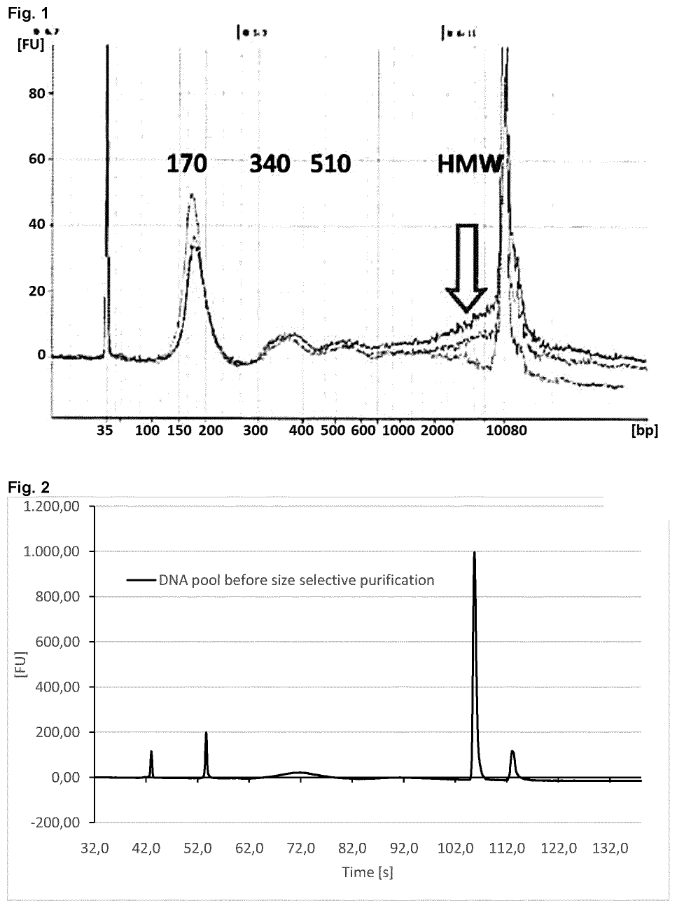

CcfDNA can be isolated from cell-free or cell-depleted body fluid samples such as e.g. blood plasma in minimal concentrations, commonly in the area of several nanogram per milliliter plasma. It is often observed that next to the fraction of mono-, di- and trinucleosomal ccfDNA peaks (with sizes of ˜170, 340, 510 bp) a high molecular weight fraction can be found in plasma. This is illustrated in FIG. 1, where nucleic acids isolated from a blood plasma sample was applied to electrophoretic separation. Three clear peaks of about 170, 340 and 510 bp in length corresponding to mono, di- and trinucleosomal extracellular cell-free DNA can be readily identified, with the mononucleosomal peak being the most prominent. Moreover, HMW species that are significantly larger, many kb larger in size, can be easily distinguished by electrophoretic separation. There is a high interest to isolate the low molecular ccfDNA fraction separately from the higher molecular weight fraction, which may either be further processed or discarded. It is of high interest to enable the specific separation of individual species of ccfDNA to derive their specific information. The below Examples show that the method according to the present disclosure may be used in order to enrich small DNA fragments, such as low molecular weight ccfDNA, in a size selective manner from a nucleic acid sample comprising small and large DNA fragments. Differently sized nucleic acid populations present in such a sample can be separated based on their size.

Throughout the Examples, DNA is first bound from the sample onto the magnetic particles comprising carboxyl groups. As is shown below, this binding step allows to capture small and larger DNA fragments. Next, the smaller DNA fraction is size-selectively eluted from the beads and can be recovered from the eluate/supernatant. This step can be repeated in order to enhance sample purification. During this “size-selective-elution” process, the longer DNA fractions remain bound to the beads. Finally, the remaining longer DNA can be eluted from the magnetic particles if desired, in order to capture the longer DNA as separate fraction.

### I. Material and Methods

### 1. Preparation of Nucleic Acid Containing Sample as Starting Material

Different sample types were analysed to assess the effectiveness of the size-selective method of the present disclosure. The used starting materials are described in the respective Examples.

### 2. Experimental Procedure

Using the method according to the present disclosure, DNA fragments having a size below and above a cut-off value were enriched from the prepared starting materials. If not indicated otherwise, the method according to the present disclosure was performed as follows:
1. Factor x times the sample volume (v/v) of PEG buffer (20% PEG 8000, 2.5 M NaCl, 1 mM EDTA, 0.05% Tween 20 (v/v), 10 mM Tris, 3.36 mM HCl) and 2 μl of carboxylated beads (inter alia M-Beads, MoBiTec, Göttingen, Germany) were added to 1 volume sample (starting material).
2. Incubation was done for 5 min at 900 rpm on a shaker to ensure DNA binding to the beads.
3. The supernatant was removed on a magnetic rack.
4. PEG buffer (v/v) was prediluted with TE buffer as specified in the examples below and added to the separated particles and mixed on a shaker at 900 rpm for 1 minute. Different dilutions were analysed as specified below for binding of DNA fragments, which therefore remain bound to the beads. Shorter DNA fragments having a size/length below the cut-off value are selectively eluted from the particles into the supernatant and may be purified therefrom.
5. Steps 3 and 4 were repeated and the supernatant removed completely. Repeating the size-selective elution of smaller DNA fragments may improve obtainment of shorter DNA fragments having a length below the cut-off value. To enhance the yield of the eluted short DNA fragments as target nucleic acids, the supernatant comprising the eluted nucleic acids obtained after each repetition may also be combined with the other obtained supernatants and the so combined supernatants may be further processed together, e.g. in order to purify the small eluted DNA fragments therefrom. Repetition of steps 3 and 4 is optional.
6. 200 μl of 80% ethanol were added to the magnetic particles and mixed in a shaker for 1 minute at 900 rpm.
7. The supernatant was removed.
8. Steps 6 and 7 were repeated.
9. The beads were air-dried for 15 minutes at RT.
10. The longer DNA was eluted from the beads in prewarmed (65° C.) 15 μl AE buffer (QIAGEN) for 3 mins at 900 rpm on a shaker.

### 3. Analytical Methods

The results of the methods were inter alia analyzed by the following methods:

### 3.1. Bioanalyzer (Agilent Genomics)

Various samples were loaded to an Agilent Bioanalyzer (Agilent High Sensitivity DNA Assay, Agilent Technologies) and the resulting electropherograms analyzed. On the electropherograms, marker bands are visible at around 42 seconds and 120 seconds.

3.2. Qubit High Sense dsDNA Kit (Invitrogen by Thermo Fisher)
3.3. Column Purification of the Supernatants Harvested from the Binding Steps and/or Size-Selective Elution Steps

For assessing the total amount of DNA that remained unbound to the magnetic particles during the binding step (a) or released from the particles during the size-selective elution step (c) according to the present invention, the harvested supernatants were column purified using the MinElute® purification kit (QIAGEN) which isolates small and large DNA from the harvested supernatants.

### II. Example 1: Size-Selection Using a DNA Sample Mimicking the Size-Distribution of Isolated Ccfdna

### 1. Starting Material

ccfDNA is commonly isolated in minimal concentrations (in the range of nanograms per ml blood plasma). Since the vast majority of the ccfDNA is around 170-510 bp long, longer nucleic acids may occur in smallest quantities, if the biological sample was properly stabilized in advance. Therefore, separating and displaying the larger molecules on a bioanalyzer is a difficult task due to their low concentrations in the sample. In order to demonstrate the feasibility of the purification protocol of the present invention, a mockup sample was generated that allows to separate DNA in the size-range of ccfDNA from higher molecular DNA components. As mockup ccfDNA sample, a combination of two DNA libraries with averages sizes of 130 bp (˜50 seconds in electropherogram) and 300 bp (˜70 seconds in electropherogram) as well as linearized pUC21 plasmid of 2.7 kb (˜105 seconds in electropherogram) was used. The sample was analyzed using a bioanalyzer. The resulting electropherogram is displayed in FIG. 2.

### 2. Experimental Procedure

The size-selective purification method which was performed in this example is described above in section 1.2.

### a) DNA Binding Using 1.6×Volumes of PEG-Buffer

Goal of the binding step (step (a)) is to bind the nucleic acids from the sample onto magnetic particles. In this configuration the bound nucleic acids can be transferred into a vessel where the size-selection will take place. In order to bind the DNA out of the sample and onto magnetic beads, 1.6×volumes of PEG buffer (see materials and method section for particular composition) were added to 1 volume DNA sample that already contained the magnetic particles. Binding was facilitated by incubation under mild agitation.

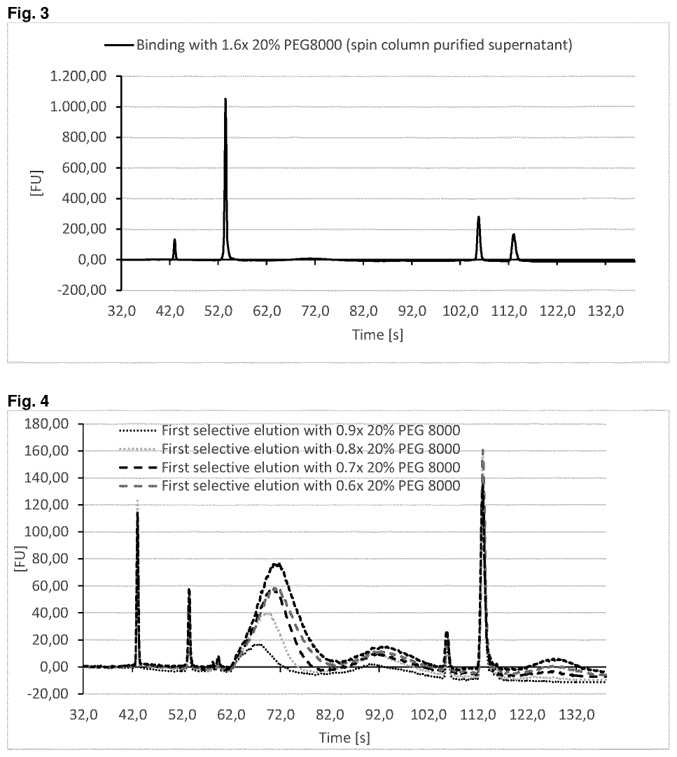

FIG. 3 shows the results of the spin-column purified supernatant of the binding step that was analyzed using a bioanalyzer in form of an electropherogram. From this supernatant, nucleic acids that were not bound in the binding step can be recovered. The electropherogram shows that predominantly all nucleic acids have been bound to the magnetic particles and were thus transferred together with the beads out of the supernatant. Although the electropherogram shows the presence of some nucleic acids in the supernatant after binding, it should be noted that the sample has been highly concentrated by the spin-column purification (260 μl supernatant was purified into 15 μl sample and analyzed without any further dilution). Also it is to be noted, that predominantly all of the nucleic acids that remained unbound to the magnetic particles belong to the fraction of small nucleic acids showing size preference for longer nucleic acids during the binding step.

### b) Size-Selective Elution of Small DNA Fragments

After binding the vast majority of DNA from the sample to the magnetic particles, the small DNA fragments (having a size below the desired cut-off value) were size-selectively eluted from the beads. The PEG buffer (see materials and method section for composition) was used in several dilutions with buffer TE to prepare different elution compositions. The supernatants of the size-selective elutions that comprise the size-selectively eluted nucleic acids were purified using a spin-column and analyzed using a bioanalyzer, in order to analyse the size-selectively eluted DNA fragments. The results are depicted in FIG. 4.

An increase in dilution of the PEG buffer leads to an increased elution of DNA. This can be seen most prominently when looking at the 300 bp (70 s) library under the influence of differentially diluted PEG buffers. When the PEG buffer is used in high concentrations (dilution factor of 0.9× and 0.8× volume PEG buffer to buffer TE), so that the PEG concentration is relatively high in the elution composition, the 300 bp library fraction has a tendency to remain bound to the magnetic particles, whereas it is readily eluted when the PEG concentration was diluted further (0.7×-0.5×). These findings indicate that for the used binding reagent, a dilution factor of 0.7 and below is optimal to ensure e.g. the desired cut-off around 500 bp in order to separate larger from smaller DNA fragments usually present in ccfDNA samples. However, for size-selective elution of even smaller ccfDNA species, it may also be beneficial to use higher dilution ratios to generate the elution composition. Moreover, the dilution ratio that is required depends on the initially applied concentration of PEG, as shown below. Therefore, a wide variety of dilution ratios, respectively PEG and salt concentrations, is applicable in the context of the method of the present disclosure.

Generally, it is important to note that under the tested conditions, short nucleic acids elute more readily from the magnetic particles compared to the longer nucleic acids, that have a tendency to remain bound to the magnetic beads. This indicates the advantageous applicability of the size-selection workflow according to the present disclosure when using ccfDNA for isolation/purification.

Moreover, the initial size selection step can be followed by additional rounds of size-selective elution if it is necessary to enhance purification/size-separation of the desired fraction. In this experiment, another round of size-selection was obtained that only resulted in minor additional elution of short DNA fragments from the sample (data not shown), substantiating the efficient size-selective elution that is already achieved in the first step (see also FIG. 11 below).

Moreover, adjusting the concentration of PEG and/or salt in a sequential step of selective elution can be performed if it is desired to isolate a nucleic acid below a further (second) cut-off value resulting from the adjusted concentrations.

c) Elution of the Longer DNA Fractions from the Magnetic Particles

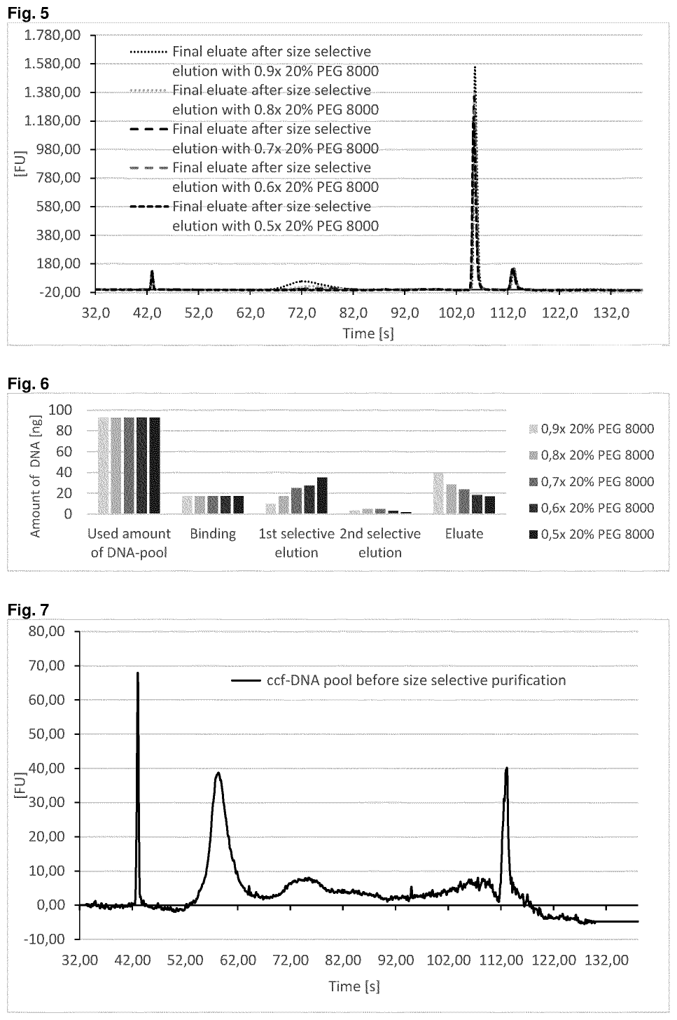

During the size-selection steps, short nucleic acids (having a size below the desired cut-off value) were selectively eluted from the magnetic particles, while larger DNAs (having a size above the cut-off value) remained bound. Therefore, the majority of the remaining nucleic acids on the beads should belong to the fraction of long nucleic acids (>500 bp) that were present in the provided sample (starting material). These longer DNA molecules may also be eluted and thus obtained in form of a separate, enriched fraction. During this optional final elution step for recovering the larger DNA fraction, the remaining DNA was eluted from the beads using an elution buffer and agitation. The DNA fragments present in the final eluates were analyzed using a bioanalyzer and the resulting electropherogram is depicted in FIG. 5.

The electropherogram depicted in FIG. 5 shows the final eluates of the size selection workflow with mockup ccfDNA. The results correlate nicely with the electropherograms shown in FIG. 4. Where the conditions for the size-selective elution favored the elution of the small 300 bp DNA (˜72 s), less DNA of that size can be detected in the final eluates, that comprise the eluted larger DNA fragments. Likewise, when the dilution of the PEG buffer was insufficient to facilitate the elution of the 300 bp library, DNA fragments of that size can be detected in the final eluates. Therefore, the dilution of the PEG buffer used for the size-selective elution step(s) (c) is a valuable parameter to tune the cut-off size and therefore, the successful separation of DNAs of different sizes. A size fractionation is possible using the method according to the present invention. As described herein, the dilution factor is interdependent with the PEG-concentration (as also shown below).

Under the chosen experimental conditions, the successful size-selective elution of nucleic acids having a size below approximately 500 bp was possible for dilutions ratios of 0.7× and lower. While a stronger dilution (e.g. 0.6×, 0.5× etc.) may enhance the elution effect, less dilution (e.g. 0.8×, 0.9×, etc.) and thus a higher PEG concentration resulted in a less efficient elution of the smaller ccfDNA species. These findings above indicate that a dilution factor for the PEG-buffer (20% PEG 8000) during the selective elution steps not exceeding 0.7× is preferred when aiming at a size-selective elution of ccfDNA having a size 600 bp or 500 bp.

### d) DNA Balancing of the Workflow

FIG. 6 shows the respective yields of each step in the size-selection workflow of the example. Please note the correlation between the size of the DNA eluted during the 1^{st} and 2^{nd} size-selection elution steps and the DNA content of the final eluates (right), which comprise the longer DNA molecules having a length above the cut-off value and which remained bound to the beads during the 1^{st} and 2^{nd} size selective elution steps. The yield of small target DNA during the size-selection steps is directly influenced by the PEG concentration. Regarding the recovered nucleic acids along the different steps in the workflow, it is important to note that the subsequent purification of the supernatant using spin-column purifications can cause significant losses of DNA during the spin-column procedure. Therefore, the quantifications of DNA during binding and selective-elution is merely a conservative estimate of the DNA that is present in this step. Nonetheless, the recovery-rates of around 75% are highly satisfactory. The yields of the method according to the present disclosure are thus advantageous.

### IV. Example 3: Size Selection Using Concentrated Ccfdna

Aim of this experiment was to prove the applicability of the novel size-selection workflow to ccfDNA, which was obtained from a biological sample.

### 1. Starting Material

As described above, ccfDNA occurs typically in minimal concentrations in biological cell-free or cell-depleted samples, often not exceeding a few nanogram of DNA per milliliter (e.g. in blood plasma). In order to perform multiple experiments on the same sample and generate good visibility of both long and short nucleic acids on the bioanalyzer, isolated ccfDNA from multiple extractions was first pooled and subsequently reduced the sample volume by using a vacuum concentrator (Eppendorf concentrator). The pooled and concentrated sample was subsequently loaded onto a bioanalyzer in order to assess the size-distribution of the starting material. The resulting electropherogram is depicted in FIG. 7. The pooled and concentrated ccfDNA contains two DNA fractions that are supposed to be separated using the size-selection workflow. A lower molecular fraction <500 bp (92 s) and a higher molecular DNA component/fraction above 500 bp.

The objective of this example was to establish a cut-off value of approximately 500 bp (˜92 seconds) at which long and short nucleic acids that are commonly present in ccfDNA samples are separated. At the same time, the method according to the present disclosure allows the user to recover both DNA-fractions from the obtained supernatants/eluates, if desired.

### 2. Experimental Procedure

The size-selective purification method was performed as described above in section 1.2. (protocol).

a) ccfDNA Binding Using 2×Volumes of PEG-Buffer

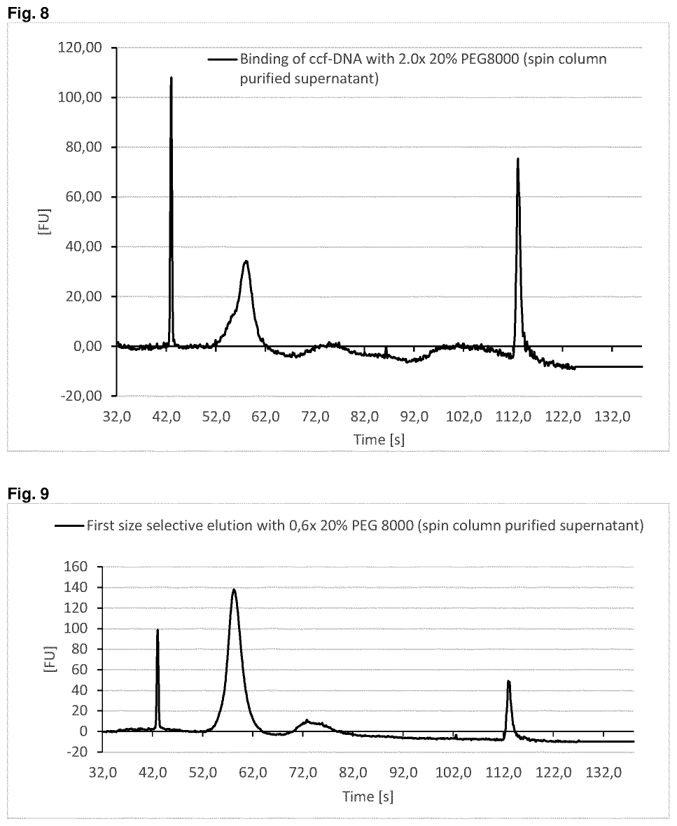

As described in the material and methods section, in a first step (step (a)) the small and large DNA present in the sample is bound to magnetic particles. Therefore, 2× the sample volume of PEG buffer was added (see material and methods section for PEG buffer composition) to 1 volume of the sample that contained the magnetic particles to generate the binding mixture of step (a). Binding took place through mild agitation on a thermal shaker. After the magnetic particles with the DNA have been removed from the cavity, the supernatant was purified using spin-columns and analyzed with a bioanalyzer. The resulting electropherogram is depicted in FIG. 8.

The increased binding factor of PEG-buffer to sample volume compared to previous experiments led to efficient DNA binding to the beads. Only a small fraction of nucleic acids remained unbound in the supernatant after binding step (a). However, it is to be noted, that the supernatant was spin-column purified and therefore heavily concentrated before the measurement (260 μl supernatant was purified into 15 μl). This means, the actual amount of DNA left unbound to the magnetic particles in binding step (a) is minor. Moreover, it seems that mostly very short nucleic acids failed to bind to the magnetic particles, while the longer DNA fraction bound to the solid phase and can be removed from the remaining sample.

Therefore, the nucleic acids that did not bind to the solid phase in step (a) and which accordingly, are still comprised in the supernatant that is obtained after the binding step predominantly consist of very small nucleic acid molecules. Hence, if all nucleic acids are to be recovered or should be recovered with a greater yield, the supernatant obtained from the binding step can be used as additional source for the size-selectively enriched small target nucleic acids having a length below the cut-off value. The supernatant obtained from the binding step may be further purified to recover the comprised small target nucleic acids. The supernatant of the binding step may be e.g. purified along with the supernatant(s) obtained from the size-selective elution step(s) (c), which comprise the size-selectively eluted target nucleic acids having a length below the cut-off value. This way it is possible to recover the entire smaller DNA-fraction from ccfDNA as enriched fraction.

b) Selective Elution of the Smaller DNAs from ccfDNA from Magnetic Beads

Aim of the size-selective elution steps are to selectively elute the smaller DNA-fraction (approximately <500 bp) from the magnetic particles while longer nucleic acids shall remain bound. Therefore, the beads are suspended into an elution composition that comprises a mixture of PEG-buffer and TE buffer in a ratio of 0.6 (×(0.6) volume PEG buffer/1 volume TE). Under these conditions the smaller DNAs are eluted from the magnetic particles into the supernatant, while the PEG concentration is sufficient to keep the longer nucleic acids bound to the beads. The size-selective elution of the nucleic acids is facilitated by mild agitation.

Following the selective-elution process, spin-columns were used to recover the eluted target DNA from the supernatant and analyzed the nucleic acids using a bioanalyzer. The resulting electropherogram is depicted in FIG. 9.

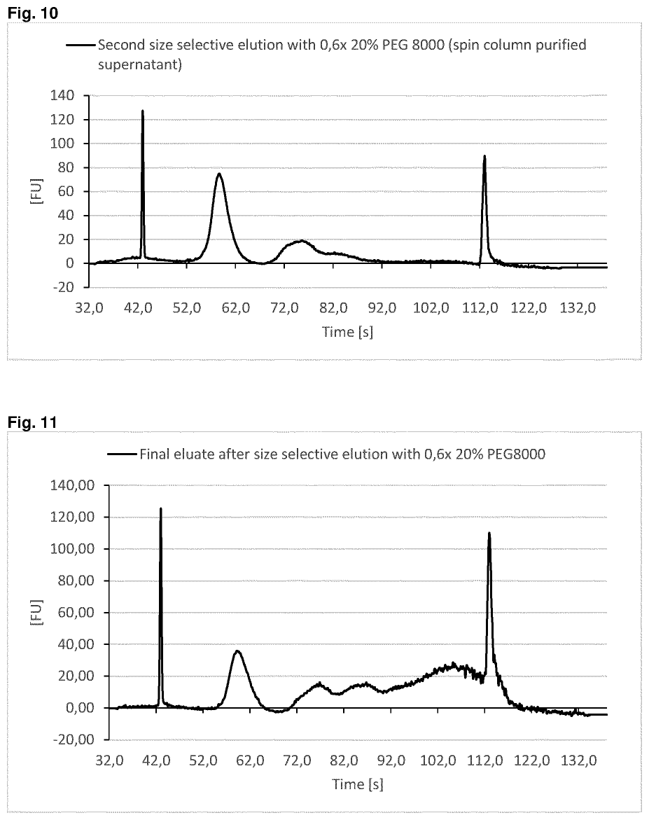

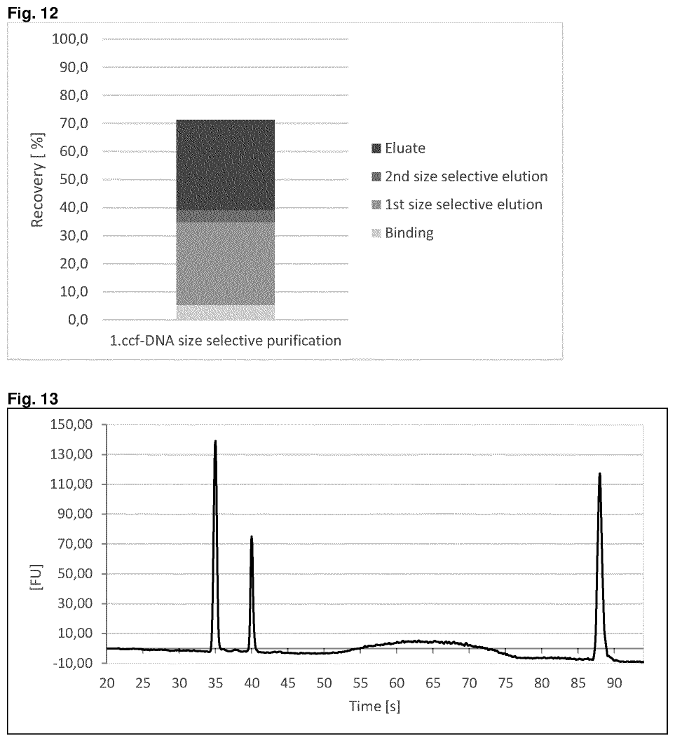

After the first size selective elution step a second round of size-selective elution was performed under the same conditions as the first. Therefore, the beads were suspended into an elution composition that comprises a mixture of PEG-buffer and buffer TE in a ratio of 0.6 PEG buffer to 1 volume TE buffer. Size-selective elution was facilitated by mild agitation of the beads. Following the selective-elution process, spin-columns were used to recover the DNA from the supernatant and analyzed the nucleic acids using a bioanalyzer. The resulting electropherogram is depicted in FIG. 10.

Comparing the electropherograms from the starting material (see FIG. 7) to the one analyzing the supernatant of the size-selective elutions (FIG. 9 and FIG. 10), it becomes apparent how well the size-selectivity of the method according to the present disclosure works. The supernatant contained almost exclusively smaller DNA fragments that were size selectively eluted, whereas the longer DNAs remained bound to the magnetic particles.

Where the electropherogram of the starting material showed a DNA peak just before the marker signal at 112 s, this DNA fraction is virtually absent in the electropherogram of the selective elution steps. This indicates that longer nucleic acids remain bound to the magnetic particles over the course of this process, while the short nucleic acids are being eluted. As described herein, these longer DNA molecules that remain bound to the beads may be eluted in a final elution step and thus recovered as separate fraction if desired.

c) Elution of the Long Nucleic Acids Comprised in ccfDNA

After the short nucleic acids have been widely depleted from the beads during the selective elution step(s), the longer nucleic acids can be eluted s well by agitating the beads in an elution buffer. The resulting final eluate was analyzed using a bioanalyzer and the resulting electropherogram is displayed in FIG. 11.

The final eluates show a wide size-distribution of DNA, including some short DNAs that remained bound to the magnetic particles during the size-selective elution procedure. However, the ratio of short nucleic acids compared to long nucleic acids has shifted heavily in favor of long DNAs, indicating that the method according to the present disclosure is applicable for ccfDNA and allows the size selective elution and thus recovery of ccfDNA fragments having a size 500 bp. Please note the presence of a very prominent long DNA fraction near the 112 s size marker. This fraction was virtually absent in the starting material (see FIG. 7) and is now highly enriched, underlining the effectiveness of the proposed workflow. In order to further improve size-selectivity it is feasible to repeat the size-selection steps multiple times.

### d) Further Conclusions

FIG. 11 shows that the method according to the present disclosure is applicable for ccfDNA and provides multiple advantages. The majority of the smaller nucleic acids having a length below the cut-off value is eluted during the size-selective elution steps (see FIG. 9 and FIG. 10), while the vast majority of the larger DNA fragments having a size above the cut-off value remains bound to the magnetic particles until the final elution step. In FIG. 11 the yields are plotted in stacked bar graphs indicating a high recovery rate of ˜70% from the starting material. As explained above, some loss due to the subsequent column based purification procedures can occur reducing the actual yield of the method according to the present disclosure. Therefore, the yield of the method according to the present disclosure can be assumed to be even higher. This is advantageous, as ccfDNA usually is present at a low concentration.

### V. Example 4: Analysis of the Effect of the Peg Concentration and Molecular Weight

The effectiveness of the selective-elution steps for size selection was shown in the previous experiments. Example 4 shows that different embodiments of the PEG-buffer are suitable, by increasing (1) the PEG concentration to 30% (w/v) and (2) changing the molecular weight of the used PEG molecules.

### 1. Preparation of Nucleic Acid Containing Sample as Starting Material

As nucleic acid containing sample, a high quality library (library A) with a size distribution varying from 300 to 1800 bp with an average fragment size of approx. 700 bp was mixed with an equal volume of a library preparation (library B) that comprised vast amounts of nucleic acids having a small length of approx. 120-130 bp. The resulting DNA containing sample provided a starting material with a high amount of small nucleic acids (see FIG. 13). The aim was to size selectively elute these smaller DNA fragments ≤150 bp during the size selective elution step, while maintaining binding of the larger DNA fragments.

### 2. Experimental Procedure

DNA fragments having a size below the cut-off value of approx. 51500 were separated from the prepared starting materials.

### Protocol

The following protocol was followed:
1. 1.1× Volume (v/v) of PEG-buffer (see below) and 4 μl of carboxylated beads (M-Beads, MoBiTec, Göttingen, Germany) were added to 1 volume sample (starting material). A higher volume could also be added to increase the PEG concentration for efficient binding of the small DNA molecules.
2. Incubation was done for 5 min at 1400 rpm on a shaker to ensure DNA binding to the beads.
3. The supernatant was removed on a magnetic rack.
4. PEG-buffer (v/v) was prediluted with TE buffer as specified in the examples below and added to the separated particles and mixed on a shaker at 900 rpm for 1 minute. Different dilutions were analysed as specified below for binding of DNA fragments, which therefore remain bound to the beads. Shorter DNA fragments having a size below the cut-off value are selectively eluted from the particles into the supernatant.
5. Steps 3 and 4 were repeated and the supernatant removed completely. Repeating the size-selective elution of smaller DNA fragments improves obtainment of shorter DNA fragments.
6. 200 μl of 80% ethanol were added and mixed in a shaker for 1 minute at 900 rpm.
7. The supernatant was removed.
8. Steps 6 and 7 were repeated.
9. The beads were air-dried for 15 minutes at RT.
10. The DNA was eluted from the beads in prewarmed (65° C.) 50 μl AE buffer (QIAGEN) for 3 mins at 1400 rpm on a shaker.

### Analytical Methods and Sample Preparation for Analysis

The results of the methods were inter alia analyzed as described above using the Agilent Bioanalyzer, Qubit High Sense dsDNA Kit and column purification.

### Example 4.1: Use of PEG 8000 in an Increased Concentration of 30% (w/v)

In this experiment the effect of 30% (w/v) PEG in the PEG-buffer on the effectiveness of the size-selection procedure was analysed. In addition to 30% (w/v) PEG 8000, all PEG-buffers contained the following components 2.5 M NaCl, 1 mM EDTA, 0.05% Tween 20 (v/v), 10 mM Tris, 3.36 mM HCl.

### a) Binding of Nucleic Acids to the Carboxylated Beads Using 1.1× Volume of PEG 8000 Buffer

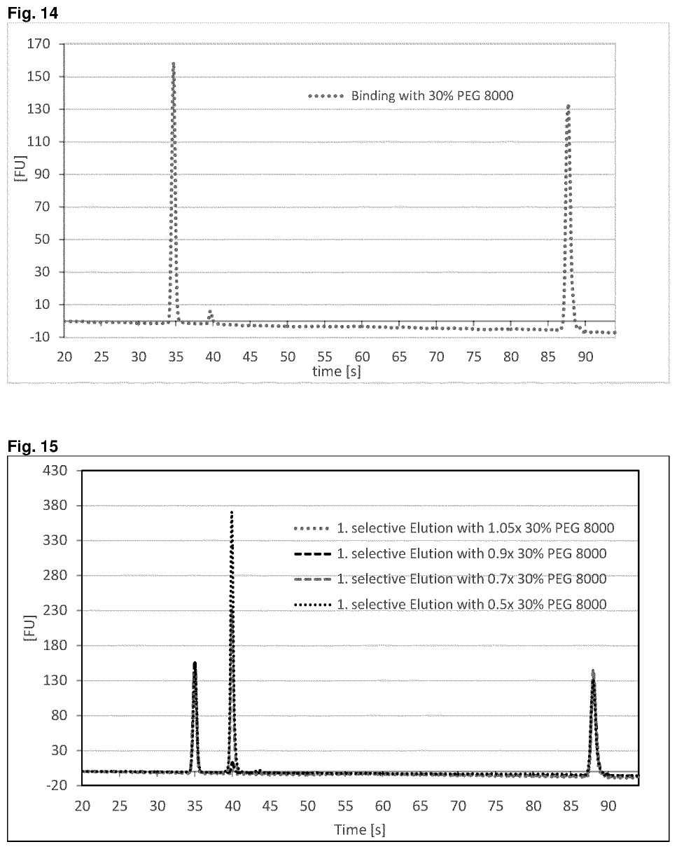

An increase in PEG concentration during the initial binding step supports binding of the larger DNA (e.g. 300 to 1800 bp and more) as well as the smaller DNA fragments (e.g. 120 to 130 bp) out of the sample onto magnetic particles. The binding dilution of the PEG-buffer is in this experiment chosen to be 1.1× volume of PEG-buffer to 1 volume sample (staring material) for binding the DNA to the magnetic particles. The remaining unbound DNA still present in the supernatant of the binding step was purified using spin-columns and analyzed using a bioanalyzer. The resulting electropherogram is depicted in FIG. 14.

The presence of PEG 8000 in high concentrations facilitates the binding of DNA to the magnetic particles as shown in FIG. 14. Using a PEG-buffer with a concentration of 30% (w/v) bound the entire DNA sample including the small DNA fragments of 120 to 130 bp to the magnetic particles. Therefore, despite using a lower dilution factor of 1.1× in comparison to the previous experiments (e.g. 1.6× and 2×), predominantly the entire DNA was bound to the solid phase, if the PEG concentration in the binding buffer was increased. In conclusion, the higher PEG concentration of 30% (w/v) influences the binding, which can allow to use lower dilution ratios of the PEG buffer. In embodiments, it is advantageous to dilute the sample less with the PEG buffer used for binding, as a higher concentration of DNA in the sample may be obtained.

b) Size-Selective Elution Using 30% (w/v) PEG-Buffer

After binding the starting material (see Example 4.a)), the magnetic particles with the bound DNA were separated and subjected to the selective-elution procedure (step (c) of the present method). Since using a PEG-buffer (reagent) with highly concentrated PEG will influence the PEG concentration during the selective elution for a given dilution factor, multiple dilutions were analyzed to assess optimal conditions for each PEG-buffer to selectively elute the DNA fraction having a size below the set cut-off value of approx. 150 bp (see FIG. 15). DNA that was eluted into the supernatant during the first selective-elution step of the protocol was spin-column purified from the supernatant and the purified DNA was analyzed using a bioanalyzer. The resulting electropherogram is shown in FIG. 15.

FIG. 15 shows an overlay of electropherograms of the spin column purified DNA from the supernatant of the 1^{st} size selective elution steps with different mixing ratios of 30% PEG 8000 (w/v) and Buffer TE as indicated in the legend (e.g. 1.05× volume PEG-buffer+1 volume TE buffer; 0.9× volume PEG buffer+1 volume TE buffer etc.). Since working with 30% PEG in the PEG buffer retrieved all DNA including the small DNA fragments (approx. 120 to 130 bp) from the starting sample during the binding step, it was expected that lower PEG concentrations and thus stronger dilutions of the 30% PEG-buffer were required in order to thoroughly elute the small DNA fragments from the beads during the size-selective elution step. In the analyzed set-up, working with a relatively low dilution factor of 1.05× volume (PEG-buffer to TE) no substantial elution of the small DNA fragments took place. Lowering the mixing ratio down to 0.7× volumes improved elution of the small DNA fragments from the beads. A complete size selective elution was achieved with a mixing ratio of 0.5× volumes. Under these conditions the larger DNA having a size of 300 to 1800 bp remained bound to the carboxylated beads, while the DNA having a size of 120 to 130 bp and thus below the cut-off value were effectively eluted into the buffer, respectively the supernatant. Hence, when the starting PEG concentration in the PEG buffer is high, higher dilutions of the PEG-buffer are necessary to achieve an efficient size-selective elution of the small DNA fraction. Noteworthy, the dilution of PEG buffer, respectively concentration of PEG depends on the desired cut-off (e.g. PEG buffer may be less diluted to increase the cut-off value).

In conclusion, FIG. 15 demonstrates the relationship of PEG concentration and selective elution: the higher the PEG concentration during the selective elution, the fewer small DNA is eluted during this step, i.e. less of the small DNA fragments are obtained.

c) Final Eluates of the Size-Selection Procedure with PEG Concentrations of 30%

The above findings were also reflected in the finally (purified) eluates obtained from the previous experiments. Desirably, the purified eluates comprise the larger DNA having a size above the cut-off value, free from the small DNA below the cut-off value. As expected, when the PEG concentration during the selective-elution steps was too high to separate the small DNA fragments of 120 to 130 bp from the remaining sample during size-selective elution, small DNA fragments were visible in the electropherograms of the final eluates that comprised the large DNA fraction. As described herein, the conditions can be adjusted to achieve that the small nucleic acid fragments (e.g. 120 to 130 bp fragments) are effectively eluted during the size selective elution steps, while the large nucleic acid fragments of 300 to 1800 bp remain bound to the beads, and may, if desired, be eluted to provide an eluate comprising the larger DNA fraction in a separate eluate.

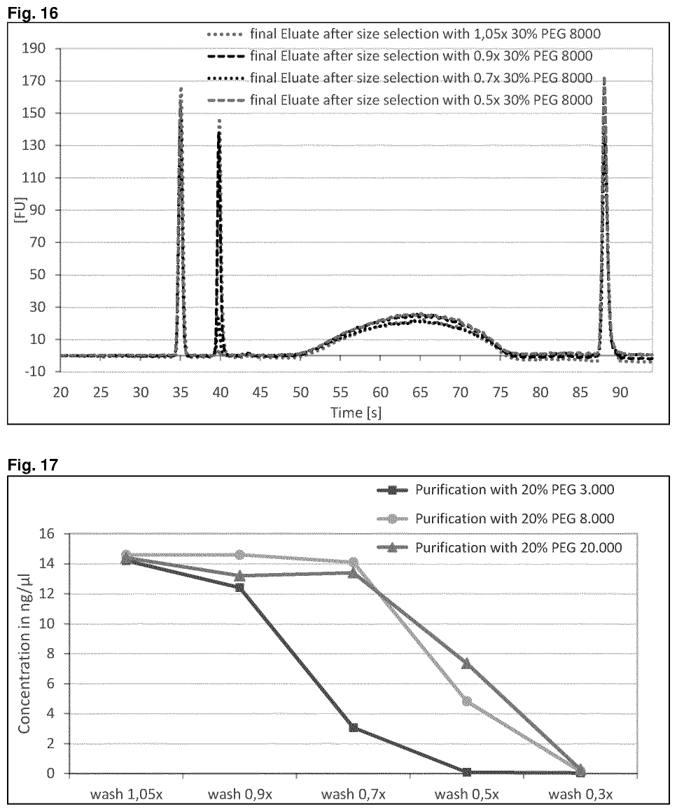

FIG. 16 shows overlaid electropherograms of the purified material (final eluates) after size selective elution with a PEG-buffer that contained 30% (w/v) PEG before being diluted with TE buffer (the dilution factor is shown in the legend). It shows how the conditions for selective elution influence the size of the DNA that remains bound to the magnetic beads and accordingly, is found in the final, purified eluates. If the PEG concentration was high during the size selective-elution, small DNA fragments were not efficiently eluted from the magnetic particles and can thus still be detected in the final eluate (e.g. dilution factor of 0.7× volume (PEG/TE) and higher). In contrast, if the PEG concentration was lower during the selective-elution step, small DNA fragments were readily eluted from the magnetic particles and are not detectable in the final eluates as visible by the flat line around 130 bp (40 s), see e.g. dilution factor of 0.5×volume PEG/TE.

In conclusion, the effective size-selective elution of the smaller nucleic acid (e.g. 120 to 130 bp) strongly depends on the PEG concentrations that are used for the size-selective elution and furthermore the initial binding step. The above experiments clearly demonstrate using PEG 8000 as an example, that a size-selective elution is possible with a wide range of PEG concentrations in the PEG-buffer. The use of highly concentrated PEG-buffers during binding whereby large and small nucleic acids are bound to the beads require a higher dilution of the PEG buffers/lower PEG concentration during the size effective elution step in order to elute substantially all the bound small DNA fragments during the size selective elution step.

### d) Calculated PEG Concentrations

As indicated above, binding of predominantly the entire DNA can be achieved by using a high volume of PEG buffer (see previous example) or by an increase of the concentration of PEG to 30% (w/v) while having a relatively low dilution factor from 1.1× sample volume. The increased concentration of PEG in the binding mixture compensates a lower dilution factor. In order to selective elute small nucleic acid fragments from the beads, high dilutions of the PEG-buffer were required during these steps. Table 1 displays the final PEG concentration during the size-selective elution steps using various dilutions factors of the 30% (w/v) PEG-buffer. It gives an overview of calculated PEG 8000 concentrations during the experimental procedure of nucleic acid separation via size-selective elution in the experimental set-up tested. Number in percent indicate the final PEG concentration during the step.

TABLE 1

| | Binding | Size | Size | Size | Size |
|---|---|---|---|---|---|
| | with | selective | selective | selective | selective |
| | factor | elution | elution | elution | elution |
| Reagent | 1.1x | 1.05x | 0.9x | 0.7x | 0.5x |
| 30% PEG | 15.7% | 15.4% | 14.2% | 12.4% | 10% |
| 8000 | | | | | |

Altogether, in the experimental set-up and for PEG 8000, the calculated concentrations of PEG in the selective-elution steps is preferably in a range between about 8% and 11% to thoroughly elute nucleic acids having a size of 120 to 130 bp from the magnetic particles while keeping the nucleic acid fraction of 300 to 1800 bp bound to the magnetic particles as indicated by bioanalyzer data (not all traces shown). Preferred conditions tested are indicated in bold in Table 1.

Therefore, the use of PEG-buffers with high PEG concentrations for binding require higher dilutions of said PEG-buffer in order to prepare the elution compositions for the size-selective elution process (in order to efficiently elute the small DNA fragments that were bound to the beads during the binding step) compared to the use of PEG-buffers with lower concentrations of PEG in order to reach the same final concentration of PEG that facilitates the selective-elution. The PEG-concentration can be adjusted to the particular nucleic acid size fraction desired. For instance, a cut-off of 500 bp (as disclosed in the above examples) required application of a different PEG concentration as the present example, which advantageously allows to selectively elute nucleic acids having a cut-off <150 bp, such as 120 to 130 bp. The disclosed method allows to adjust the concentration of PEG and salt in order to set a desired cut-off value and then separate nucleic acids having a length below (selective elution) and above (final eluate) the cut-off value by the size-selective elution steps.

### Example 4.2: Use of PEG with Different Molecular Weights

Polyethylene glycol (PEG) with varying molecular weight was used to investigate the influence of the molecular weight on the method according to the present disclosure. Since PEG functions as a molecular crowding agent, it is believed that the length of the PEG chains, which corresponds to its molecular weight, would directly influence the concentration of PEG that is needed to successfully perform the method of the invention. The processing of samples and the starting material has been described above. One of the following PEG molecules was used in the PEG-buffer for these experiments: PEG 3000, PEG 8000 or PEG 20000. Apart from 20% PEG, the buffer contained 2.5 M NaCl, 1 mM EDTA, 0.05% Tween 20 (v/v), 10 mM Tris and 3.36 mM HCl.

A successful separation of nucleic acids based on their size is possible using polyethylene glycol (PEG) of varying molecular weight. In this experiment the performance of three different PEG molecules was tested: PEG 3000, 8000 and 20000. All of these tested PEGs variations mediated the binding of the starting material to the magnetic particles. Moreover, the various sized PEG molecules allow to size-selectively elute the nucleic acids. Here, longer PEG molecules seem to facilitate maintaining binding of DNA having a size above the cut-off value to the magnetic particles during the size selective elution process. When using a PEG with lower molecular weight, the point of selectively eluting larger nucleic acids is already reached in lower PEG dilutions, indicating that the length of the PEG (molecular weight) influences that DNA binding to magnetic particles is maintained during size selective elution. A conclusive plot of the yields following the procedures is shown in FIG. 17.

### FURTHER CONCLUSIONS

The results depicted in FIG. 17 show that differences of the molecular weight of the used PEG can be utilized to adapt the binding and selective elution conditions of nucleic acids to the solid phase. Accordingly, the graph supports that PEG polymers of different molecular weight may be used. It furthermore shows that changing the molecular weight throughout the steps of the method according to the present disclosure allows to achieve efficient nucleic acid separation based on size. For instance, applying a PEG having a high molecular weight during in the binding mixture of binding step (a), followed by applying a PEG having a lower molecular weight in the elution composition of step (c) allows to selectively elute nucleic acids having a length below the cut-off value. Additionally, the concentration of the PEG may be changed (e.g., reduced in step (c) in order to further modify the cut-off value for selective elution.

1. A poly(alkylene oxide) polymer based size selective method for enriching nucleic acid molecules having a length below a cut-off value from a nucleic acid containing sample, the method comprising:
(a) preparing a binding mixture comprising
the nucleic acid containing sample,
a poly(alkylene oxide) polymer and
a salt
and binding nucleic acid molecules of different sizes to a solid phase which comprises a functional group, preferably carboxylated magnetic particles;
(b) separating the solid phase with the bound nucleic acid molecules from the remaining sample; and
(c) contacting the solid phase with the bound nucleic acid molecules at least once with an elution composition comprising a poly(alkylene oxide) polymer and a salt to selectively elute nucleic acid molecules having a length below the cut-off value from the solid phase while larger nucleic acid molecules having a length above the cut-off value remain bound to the solid phase,
wherein the concentration (w/v) of the poly(alkylene oxide) polymer in the elution composition is lower than the concentration (w/v) of the poly(alkylene oxide) polymer in the binding mixture of (a);
(d) separating the solid phase with the bound larger nucleic acid molecules from the eluted nucleic acid molecules; and
(e) optionally further purifying the eluted nucleic acid molecules.

2. The method according to claim 1, wherein the nucleic acid containing sample is a cell-free or cell-depleted body fluid sample and wherein the eluted nucleic acid molecules are extracellular nucleic acid molecules, preferably extracellular DNA.

3. The method according to claim 1 or 2, wherein the cut-off value fulfills at least one of the following characteristics:
(i) a cut-off value is established in step (c) so that at least nucleic acid molecules having a length of 2000 nt remain bound to the solid phase;
(ii) a cut-off value is established in step (c) so that at least nucleic acid molecules having a length of 1500 nt remain bound to the solid phase;
(iii) a cut-off value is established in step (c) so that at least nucleic acid molecules having a length of 1000 nt remain bound to the solid phase;
(iv) a cut-off value is established in step (c) so that at least nucleic acid molecules having a length of 800 nt remain bound to the solid phase;
(v) a cut-off value is established in step (c) so that at least nucleic acid molecules having a length of 600 nt remain bound to the solid phase;
(vi) a cut-off value is established in step (c) so that at least nucleic acid molecules having a length of 500 nt remain bound to the solid phase.

4. The method according to any one of claims 1 to 3, having one or more of the following characteristics:
(i) a cut-off value is established in step (c) by adjusting the concentration (w/v) of the poly(alkylene oxide) polymer and optionally the salt in the elution composition so that at least nucleic acid molecules having a length of 350 nt are eluted from the solid phase;
(ii) a cut-off value is established in step (c) by adjusting the concentration of the poly(alkylene oxide) polymer and optionally the salt in the elution composition so that at least nucleic acid molecules having a length of 500 nt or 600 nt are eluted from the solid phase;
(iii) a cut-off value is established in step (c) by adjusting the concentration of the poly(alkylene oxide) polymer and optionally the salt in the elution composition so that nucleic acid molecules having a length of 600 nt are eluted from the solid phase while larger nucleic acid molecules remain bound to the solid phase;
(iv) a cut-off value is established in step (c) by adjusting the concentration of the poly(alkylene oxide) polymer and optionally the salt in the elution composition so that nucleic acid molecules having a length of <500 nt are eluted from the solid phase while larger nucleic acid molecules remain bound to the solid phase;
(iv) size selective elution performed in step (c) provides an eluted fraction of nucleic acid molecules wherein the majority of the nucleic acid molecules comprised in the eluted fraction have a length 2000 nt, such as 1500 nt, 1000 nt, 800 nt, 700 nt, 600 nt, or 500 nt, depending on the selected cut-off value.

5. The method according to one or more of claims 1 to 4, further comprising
(f) eluting nucleic acid molecules having a length above the cut-off value from the solid phase that was separated in step (d), wherein optionally, the eluted nucleic acid molecules are further purified.

6. The method according to one or more of claims 1 to 5, wherein the solid phase has one or more of the following characteristics:
a) the solid phase comprises ionic groups, preferably acidic groups as functional group;
b) the solid phase comprises carboxyl groups as functional group;
c) the solid phase is provided by particles, preferably magnetic particles; and/or
d) the solid phase is provided by carboxylated magnetic particles.

7. The method according to one or more of claims 1 to 6, having one or more of the following characteristics
i) the poly(alkylene oxide) polymer is a polyethylene glycol;
ii) the poly(alkylene oxide) polymer, which preferably is a polyethylene glycol, has a molecular weight that lies in a range of 2000 to 40000, preferably in a range selected from 3000 to 30000 and 5000 to 25000, such as in a range of 6000 to 20000;
iii) a poly(alkylene oxide) polymer, preferably a polyethylene glycol, of the same molecular weight is used in step (a) and in step (c); or
a poly(alkylene oxide) polymer, preferably a polyethylene glycol, of differing molecular weight is used in step (a) and in step (c), wherein if the molecular weight differs, the molecular weight of the polymer that is used in step (c) is higher or lower, preferably higher, than the molecular weight of the polymer that is used in step (a);
iv) the binding mixture comprises the poly(alkylene oxide) polymer, which preferably is a polyethylene glycol, in a concentration of at least 8% (w/v), preferably at least 9%, at least 10%, at least 11% or at least 12%; and/or
v) the binding mixture comprises the poly(alkylene oxide) polymer, which preferably is a polyethylene glycol, in a concentration that lies in a range of 8% to 30% (w/v), preferably in a range selected from 9% to 25% (w/v), 10% to 20% (w/v), 11% to 18% (w/v) and 12% to 15% (w/v).

8. The method according to one or more of claims 1 to 7, having one or more of the following characteristics:
i) the salt is a non-chaotropic salt;
ii) the salt is a monovalent salt;
iii) the salt is an alkali metal salt, preferably an alkali metal halide;
iv) the salt is a chloride salt, preferably selected from sodium chloride, potassium chloride, lithium chloride and cesium chloride, more preferably the salt is sodium chloride;
v) the salt is present in the binding mixture in a concentration of 500 mM, optionally selected from 750 mM, 1M, 1.25M and 1.5M; and/or
vi) the salt is present in the binding mixture in a concentration that lies in a range of 500 mM to 4M, optionally selected from 750 mM to 3.5M, 1M to 3M, 1.25M to 2.5M, and 1.5M to 2M.

9. The method according to one or more of claims 1 to 8, wherein step (a) comprises adding a binding reagent to the nucleic acid containing sample to prepare the binding mixture, wherein the binding reagent comprises the poly(alkylene oxide) polymer, preferably a polyethylene glycol, and the salt.

10. The method according to claim 9, having one or more of the following characteristics:
a) the binding conditions are exclusively established by contacting the nucleic acid containing sample with the binding reagent;
b) the binding reagent comprises the salt, which preferably is an alkali metal salt, in a concentration that lies in a range of 0.5M to 5M, preferably in a range selected from 0.7M to 4.5M, 1M to 4.25M, 1.25M to 4M, 1.5M to 3.75M and 1.75M to 3.5M;
c) the binding reagent comprises the poly(alkylene oxide) polymer, which preferably is a polyethylene glycol, in a concentration that lies in a range of 10% to 50% (w/v), preferably in a range selected from 11% to 45%, 12% to 40% and 15% to 35% (w/v);
d) the binding reagent is selected from the group of the following binding reagents:
(aa) a binding reagent comprising
a polyethylene glycol having a molecular weight that lies in a range of 2000 to 40000, preferably in a range selected from 3000 to 30000, 5000 to 25000 and 6000 to 25000;
an alkali metal salt in a concentration that lies in a range of 0.5M to 5M, preferably selected from 0.7M to 4.5M, 1M to 4.25M, 1.25M to 4M, 1.5M to 3.75M and 1.75M to 3.5M;
(bb) a binding reagent comprising
a polyethylene glycol having a molecular weight that lies in a range of 3000 to 30000, preferably in a range selected from 5000 to 25000 and 6000 to 20000;
an alkali metal salt in a concentration that lies in a range of 1M to 4M, preferably selected from 1.5M to 3.75M and 2M to 3.5M;
(cc) a binding reagent comprising
a polyethylene glycol having a molecular weight that lies in a range of 5000 to 25000, optionally 6000 to 20000, in a concentration that lies in a range of 10% to 45% (w/v), preferably selected from 11% to 40% (w/v), 12% to 35% (w/v) and 15% to 30% (w/v);
an alkali metal salt in a concentration that lies in a range of 1M to 4M, preferably selected from 1.5M to 3.75M and 2M to 3.5M;
and/or
(dd) a binding reagent comprising
a polyethylene glycol having a molecular weight that lies in a range of 5000 to 25000, optionally 6000 to 20000, in a concentration that lies in a range of 12% to 40% (w/v), preferably 15% to 35% (w/v);
an alkali metal salt chloride, preferably selected from sodium chloride and potassium chloride, in a concentration selected from 1.5M to 3.5M and 2M to 3M; and/or
e) the binding reagent comprises the solid phase which is provided by particles, preferably magnetic particles.

11. The method according to one or more of claims 1 to 10, wherein the elution composition of step (c), which preferably is provided by a single reagent (c), has one or more of the following characteristics:
a) it comprises the poly(alkylene oxide) polymer, which preferably is a polyethylene glycol, in a concentration of at least 5% (w/v);
b) it comprises the poly(alkylene oxide) polymer, which preferably is a polyethylene glycol, in a concentration that lies in a range of 5% to 15% (w/v), preferably in a range selected from 5.5% to 12%, 6% to 11%, 6.25% to 10%, 6.5% to 9% and 6.5 to 8.5 (w/v);
c) it comprises the salt, which preferably is an alkali metal salt, more preferably sodium chloride, in a concentration of 350 mM, preferably selected from 500 mM, 700 mM and 750 mM;
d) it comprises the salt, which preferably is an alkali metal salt, more preferably sodium chloride, in a concentration that lies in a range of 350 mM to 3.5M, preferably in a range selected from 500 mM to 3M, 600 mM to 2.5M, 700 mM to 2M, 725 mM to 1.5M and 750 mM to 1.25M; and/or
e) the elution composition of step (c) is selected from the group of the following reagents:
(aa) an elution reagent composition (c) comprising
a polyethylene glycol having a molecular weight that lies in a range of 3000 to 40000, preferably in a range selected from 3000 to 30000, 5000 to 25000, 6000 to 25000 and 8000 to 20000; and
an alkali metal salt in a concentration that lies in a range of 350 mM to 3.5M, preferably in a range selected from 500 mM to 3M, 600 mM to 2.5M, 650 mM to 2M, 700 mM to 1.5M and 750 mM to 1.25M;
(bb) an elution reagent composition (c) comprising
a polyethylene glycol having a molecular weight that lies in a range selected from 5000 to 25000, such as in a range of 6000 to 25000 or 8000 to 20000; and
an alkali metal salt in a concentration that lies in a range of 500 mM to 2.5M, preferably in a range selected from 600 mM to 2M, 700 mM to 1.5M and 750 mM to 1.15M;
(cc) an elution reagent composition (c) comprising
a polyethylene glycol having a molecular weight that lies in a range of 5000 to 25000, such as in a range of 6000 to 25000 or 8000 to 20000, in a concentration that lies in a range of 5% to 12% (w/v), preferably in a range selected from 5.5% to 10%, 6% to 9% and 6.5% to 8.5% (w/v), and
an alkali metal salt in a concentration of 500 mM to 2.5M, preferably selected from 600 mM to 2M, 700 mM to 1.5M and 750 mM to 1.15M; and
(dd) an elution reagent composition (c) comprising
a polyethylene glycol having a molecular weight that lies in a range of 5000 to 25000, such as in a range of 6000 to 25000 or 8000 to 20000, in a concentration that lies in a range of 5.5% to 10% (w/v), such as 6.0% to 9% and 6.5% to 8.5% (w/v), and
an alkali metal salt chloride, preferably selected from sodium chloride and potassium chloride, in a concentration selected from 650 mM to 1.5M, 700 mM to 1.25M and 750 mM to 1.15M.

12. The method according to one or more of claims 9 to 11, comprising providing the elution composition of step (c) by diluting the binding reagent with a dilution solution, optionally wherein the binding reagent is a binding reagent having one or more characteristics as defined in claim 9 or 10 and the provided elution composition of step (c) has one or more characteristics as defined in claim 11 a) to e).

13. The method according to one or more of claims 1 to 12, wherein the nucleic acid containing sample has one or more of the following characteristics:
(i) it is a cell-free or cell-depleted biological sample, preferably a cell-free or cell-depleted body fluid sample;
(ii) it is selected from the group consisting of body fluids, body secretions, nasal secretions, vaginal secretions, wound secretions and excretions, preferably selected from blood, plasma, serum, urine, saliva, lymphatic fluid, liquor, ascites, milk, bronchial lavage, sputum, amniotic fluid, semen/seminal fluid, wherein preferably, cells were depleted from the sample prior to isolating extracellular nucleic acid molecules having a length below the cut-off value from the obtained cell-free or cell-depleted sample;
(iii) it is selected from plasma, serum, urine, saliva and/or liquor;
(iv) it is plasma or urine;
(v) it is a digested sample; and/or
(vi) it is a stabilized sample.

14. The method according to any one of claims 1 to 13, characterized by the following features:
the method is for enriching extracellular nucleic acid molecules comprised in a cell-free or cell-depleted body fluid according to their size,
the nucleic acid containing sample is a cell-free or cell-depleted body fluid sample,
step (a) comprises adding a binding reagent to the nucleic acid containing sample, which is a cell-free or cell-depleted body fluid sample, to prepare the binding mixture, wherein the binding reagent comprises the poly(alkylene oxide) polymer, preferably a polyethylene glycol, and the salt,
a cut-off value is established in step (c) by adjusting the concentration (w/v) of the poly(alkylene oxide) polymer and optionally the salt concentration in the elution composition so that at least nucleic acid molecules having a length of 350 nt are eluted from the solid phase while at least nucleic acid molecules having a length of 1000 nt remain bound to the solid phase, and
the eluted nucleic acid molecules comprise extracellular nucleic acid molecules, preferably extracellular DNA.

15. The method according to claim 14, wherein the cut-off value that is established in step (c) by adjusting the concentration of the poly(alkylene oxide) polymer and optionally the salt concentration in the elution composition is such that at least nucleic acid molecules having a length of 500 nt or 600 nt are eluted from the solid phase.

16. The method according to claim 14 or 15, comprising providing the elution composition of step (c) by diluting the binding reagent with a dilution solution, optionally wherein the binding reagent is a binding reagent having one or more characteristics as defined in claim 10 and the provided elution composition of step (c) has one or more characteristics as defined in claim 11 a) to e).

17. The method according to claim 16, wherein the solid phase with the bound nucleic acid is in step (c) only contacted with a single elution composition (c), but not with further reagents, such as further solutions.

18. The method according to any one of claims 14 to 17, wherein the binding reagent has a pH value that lies in a range of 4.5 to 9.5, such as 5 to 9 or 7 to 8.5 and wherein the solid phase is provided by carboxylated magnetic particles.

19. The method according to one or more of claims 1 to 18 for enriching target extracellular DNA molecules having a length below a cut-off value from a cell-depleted or cell-free body fluid sample, wherein the method comprises
(a) preparing a binding mixture comprising
the cell-depleted or cell-free body fluid sample, which optionally is a digested sample,
a polyethylene glycol in a concentration of at least 10%, preferably in a range of 12% to 25%, wherein the polyethylene glycol has a molecular weight that lies in a range of 3000 to 30000, preferably in a range of 5000 to 25000, and
the salt in a concentration of 750 mM, preferably at least 1M, wherein the salt is an alkali metal salt, preferably a non-chaotropic alkali metal salt, more preferably selected from sodium chloride and potassium chloride,
and binding nucleic acid molecules of different sizes to the solid phase, wherein the solid phase is provided by carboxylated magnetic particles and the bound nucleic acids include the target extracellular DNA molecules;
(b) separating the solid phase with the bound nucleic acid molecules from the remaining sample;
(c) contacting the solid phase with the bound nucleic acid molecules at least once with an elution composition to selectively elute the target extracellular DNA having a size below the set cut-off value from the solid phase while DNA molecules having a size above the cut-off value remain bound to the solid phase, wherein the elution composition comprises
a polyethylene glycol having a molecular weight that lies in a range of 3000 to 30000, preferably in a range of 5000 to 25000, in a concentration that lies in a range from 5% to 10%, preferably 6% to 9% or 6.5% to 8.5% (w/v);
the salt in a concentration of at least 500 mM, preferably 750 mM wherein the salt is an alkali metal salt, preferably a non-chaotropic alkali metal salt, more preferably selected from sodium chloride and potassium chloride, and
wherein the concentration (w/v) of the polyethylene glycol in the elution composition is lower than the concentration (w/v) of the polyethylene glycol in the binding mixture of (a); and
(d) separating the solid phase with the bound larger nucleic acid molecules from the eluted extracellular DNA molecules;
the method optionally further comprising steps (e) and/or (f)
(e) further purifying the eluted target extracellular DNA molecules;
(f) eluting nucleic acid molecules having a length above the cut-off value from the solid phase that was separated in step (d).

20. The method according to claim 19, wherein the method is for enriching extracellular DNA molecules having a length below a cut-off value of 1000 nt, 800 nt or 600 nt from larger DNA molecules comprised in the cell-free or cell-depleted body fluid sample.

21. The method according to claim 19 or 20, wherein the eluate that is provided as result of performing steps (c) and (d) comprises predominantly extracellular DNA molecules having a length 800 nt or preferably 600 nt.

22. A method for enriching target extracellular DNA molecules having a length below a cut-off value from a cell-depleted or cell-free body fluid sample, comprising enriching target extracellular DNA molecules from the sample using the method as defined in any one of claims 1 to 21.

23. The method according to claim 22, wherein the method comprises
(a) contacting a binding reagent that comprises polyethylene glycol as poly(alkylene oxide) polymer and a salt with a cell-free or cell-depleted body fluid sample, which optionally is a digested sample, thereby preparing a binding mixture comprising
the sample,
polyethylene glycol in a concentration that lies in a range of 10% to 25% (w/v), preferably 12% to 20% (w/v), wherein the polyethylene glycol has a molecular weight that lies in a range of 3000 to 30000, preferably in a range of 5000 to 25000, and
the salt in a concentration of ≥1M, wherein the salt is an alkali metal salt, preferably a non-chaotropic alkali metal salt chloride, more preferably selected from sodium chloride and potassium chloride,
and binding nucleic acid molecules of different sizes to the solid phase which comprises carboxyl groups as functional group, wherein the solid phase is preferably provided by carboxylated magnetic particles, and the bound nucleic acids include the target extracellular DNA molecules;
(b) separating the solid phase with the bound nucleic acid molecules from the remaining sample;
(c) contacting the solid phase with the bound nucleic acid molecules at least once with an elution composition to selectively elute the target extracellular DNA molecules having a size below the cut-off value from the solid phase while DNA molecules having a size above the cut-off value remain bound to the solid phase, wherein the elution composition comprises
a polyethylene glycol having a molecular weight that lies in a range of 3000 to 30000, preferably in a range of 5000 to 25000, in a concentration that lies in a range from 5% to 10%, preferably 6% to 9%, more preferably 6.5% to 8.5% (w/v);
the salt in a concentration of at least 500 mM, preferably 750 mM wherein the salt is an alkali metal salt, preferably a non-chaotropic alkali metal salt chloride, more preferably selected from sodium chloride and potassium chloride, and
wherein the concentration (w/v) of the polyethylene glycol in the elution composition is lower than the concentration (w/v) of the polyethylene glycol in the binding mixture of (a) and wherein the concentration of the salt in the elution composition is lower than the concentration of the salt in the binding mixture of (a), optionally wherein the elution composition is provided by diluting the binding reagent used in step (a) with a dilution solution; and
(d) separating the solid phase with the bound larger DNA molecules from the eluted target extracellular DNA molecules;
the method optionally further comprising steps (e) and/or (f)
(e) further purifying the eluted extracellular DNA molecules;
(f) eluting DNA molecules having a length above the cut-off value from the solid phase that was separated in step (d).

24. The method according to any one of claims 1 to 23, wherein the eluted nucleic acids comprise extracellular nucleic acid molecules, preferably extracellular DNA, and the method comprises analyzing the eluted extracellular nucleic acid molecules to identify, detect, screen for, monitor or exclude a disease, an infection and/or at least one fetal characteristic.

25. A kit for the size selective enrichment of nucleic acid molecules, preferably extracellular DNA molecules, having a length below a cut-off value from a nucleic acid containing sample, comprising
(a) a binding reagent comprising at least one poly(alkylene oxide) polymer and at least one salt;
(b) magnetic particles for binding target nucleic acid molecules in the presence of the binding reagent (a); and
(c) an elution reagent comprising at least one poly(alkylene oxide) polymer and at least one salt and/or a dilution reagent for preparing the reagent (c) by combining the dilution reagent with the binding reagent;
(d) optionally at least one washing solution; and
(e) optionally an elution solution,
wherein the concentration of the poly(alkylene oxide) polymer in the binding reagent (a) is higher than the concentration of the poly(alkylene oxide) polymer in the reagent (c).

26. The kit according to claim 25, having one or more of the following characteristics:
a) wherein the concentration of the salt in the binding reagent (a) is higher than the concentration of the salt in the reagent (c);
b) the binding reagent (a) has one or more characteristics as defined in claim 9;
c) reagent (c) has one or more of the characteristics as defined in claim 10;
d) the solid phase has one or more of the characteristics as defined in claim 3; and/or
e) the solid phase is comprised in binding reagent (a), wherein preferably, the magnetic particles are carboxylated magnetic particles.
